SEQUENCE LISTING (20 sequences)

SEQ ID NO: 1 - DNA, 22 bp, Artificial sequence
  Single strand DNA oligonucleotide
ggttcttacttcaggggccatc

SEQ ID NO: 2 - DNA, 22 bp, Artificial sequence
  Single strand DNA oligonucleotide
gtggaattttctgagggtaggc

SEQ ID NO: 3 - DNA, 22 bp, Artificial sequence
  Double stranded DNA oligonucleotide probe for EMSA
agttgaggggactttcccaggc

SEQ ID NO: 4 - DNA, 22 bp, Artificial sequence
  Double stranded DNA oligonucleotide probe for EMSA
attcgatcggggcggggcgagc

SEQ ID NO: 5 - DNA, 18 bp, Artificial sequence
  Single strand DNA oligonucleotide
atgttgtgtcgggcggcg

SEQ ID NO: 6 - DNA, 24 bp, Artificial sequence
  Single strand DNA oligonucleotide
aggtagtaagcgtgctcccacacg

SEQ ID NO: 7 - DNA, 20 bp, Artificial sequence
  Single strand DNA oligonucleotide
ttcaaggccacagatgtgcc

SEQ ID NO: 8 - DNA, 21 bp, Artificial sequence
  Single strand DNA oligonucleotide
tcgggcaatggtcttgtaggc

SEQ ID NO: 9 - DNA, 19 bp, Artificial sequence
  Single strand DNA oligonucleotide
tccgccccttccgctgatg

SEQ ID NO: 10 - DNA, 21 bp, Artificial sequence
  Single strand DNA oligonucleotide
cacggaaggccatgccagtga

SEQ ID NO: 11 - DNA, 20 bp, Artificial sequence
  Single strand DNA oligonucleotide
ggaggcaagcataagactgg

SEQ ID NO: 12 - DNA, 20 bp, Artificial sequence
  Single strand DNA oligonucleotide
gtctgccctttctcccttct

SEQ ID NO: 13 - DNA, 20 bp, Artificial sequence
  Single strand DNA oligonucleotide
acctttggagaatgtggtgc

SEQ ID NO: 14 - DNA, 20 bp, Artificial sequence
  Single strand DNA oligonucleotide
gtgatggtacgagatgggct

SEQ ID NO: 15 - DNA, 20 bp, Artificial sequence
  Single strand DNA oligonucleotide
ggaagagcaaatggctgaag

SEQ ID NO: 16 - DNA, 20 bp, Artificial sequence
  Single strand DNA oligonucleotide
agaacttcacaaacccgcac

SEQ ID NO: 17 - DNA, 20 bp, Artificial sequence
  Single strand DNA oligonucleotide
cggctaccacatccaaggaa

SEQ ID NO: 18 - DNA, 22 bp, Artificial sequence
  Single strand DNA oligonucleotide
cgctattggagctggaattacc

SEQ ID NO: 19 - DNA, 20 bp, Artificial sequence
  Single strand DNA oligonucleotide
gccatcaacgaccccttcat

SEQ ID NO: 20 - DNA, 20 bp, Artificial sequence
  Single strand DNA oligonucleotide
ttcacacccatcacaaacat

(19) United States
(12) Patent Application Publication
(10) Pub. No.: US 2007/0026090 A1
(43) Pub. Date: Feb. 1, 2007

(54) Treatment and prevention of inflammatory disease and mitochondrial dysfunction with high dose selenium

(76) Inventors: Oren Tirosh, Nes Ziona (IL); Ram Reifen, Rehovot (IL)

Correspondence Address: Martin D Moynihan;Prtsi Inc, PO Box 16446, Arlington, VA, 22215, US

(21) Appl. No.: 10/570,789

(22) PCT Filed: Jul. 29, 2004
(86) PCT No.: PCT/IL04/00700

§ 371 Date: Aug. 16, 2006

Related U.S. Application Data
(60) Provisional application No. 60/500,255, filed on Sep. 5, 2003.
(60) Provisional application No. 60/532,574, filed on Dec. 29, 2003.

(51) Int. Cl. A61K 33/04; A61K 38/05; A61K 31/198; A61K 31/195; A61K 31/095

(52) U.S. Cl. 424/702; 514/561; 514/706; 514/18

(57) ABSTRACT

The present invention is of a method for reducing activity of inflammatory immune cells by exposure to selenium. Particularly, the invention relates to a method of short-term, high dose dietary selenium supplementation for treating a subject having an inflammatory disease, particularly inflammatory bowel disease (IBD). An article of manufacture comprising a pharmaceutical composition providing a therapeutic selenium unit dosage suitable for the method of the present invention is also provided.

### FIELD AND BACKGROUND OF THE INVENTION

The present invention relates to a novel high selenium dosing for downregulation of inflammatory cell activity and treatment and/or prevention of inflammatory disease. Particularly, there is disclosed a short-term dietary supplementation with high doses of selenite, leading to selenite enrichment of colon tissue and prevention of mitochondrial dysfunction, colon necrosis, and inflammation.

The term “Inflammatory Bowel Disease” is commonly used to refer to a group of related, yet distinct chronic inflammatory conditions affecting the gastrointestinal tract. Diagnosis of these chronic inflammatory disorders is commonly made into one of three groups, Crohn's disease (CD), ulcerative colitis (UC) and undefined colitis, digestive disorders of complex pathogenesis [Podolsky, D. (2000a). Pride and prejudice: inflammatory bowel disease models and drug development, current opinions in gastroenterology 16, 295-6. Podolsky, D. K. (2000b). Going the Distance—The Case for True Colorectal-Cancer Screening, N Engl J Med 343, 207-208.] characterized by relapses and spontaneous or therapy-induced remissions. Crohn's disease may involve any segment of the gastrointestinal tract, although characteristically the region of greatest involvement is the small bowel and proximal colon, with occasional involvement of stomach, esophagus and duodenum. In Crohn's disease, the lesions are usually described as extensive linear fissures. In ulcerative colitis the inflammation is, by definition, limited to the mucosa of the large bowel. IBD may lead to serious gastrointestinal as well as extra intestinal complications, such as involvement of the hepatobiliary, cardiovascular and neural systems [Lossos, A. et al. (1995). Neurologic aspects of inflammatory bowel disease, Neurology 45, 416-21; Wewer, V., et al. (1991). Prevalence of hepatobiliary dysfunction in a regional group of patients with chronic inflammatory bowel disease, Scand J Gastroenterol 26, 97-102]. There is an increasing prevalence of IBD throughout the world, especially in the Western World. Presently, over 1 million people in America and an additional one million in Europe suffer from IBD and its symptoms which include diarrhea, loss of appetite, joint pains, ano-rectal sores, rectal bleeding and fistulas (CCFA, 1999).

At present the exact etiology of IBD remains unknown, and the initial lesion has not been clearly defined; however, patchy necrosis of the surface epithelium, focal accumulations of leukocytes adjacent to glandular crypts, and an increased number of intraepithelial lymphocytes and certain macrophage subsets have been described as putative early changes, especially in Crohn's disease. Thus, infection and immunogenic agents have been implicated in the disease process. In active IBD, there is an increased local production of proinflammatory cytokines, synthesis of eicosanoids and recruitment of both immunologically specific, and non-specific inflammatory cells from the circulation [Nicholls, R. J. (2002). Review article: ulcerative colitis—surgical indications and treatment, Aliment Pharmacol Ther 16 Suppl 4, 25-8].

Although medical therapeutics and surgery have improved the prognoses of IBD over the last two decades, there is no cure for the disease [Moum, B. (2000). Medical treatment: does it influence the natural course of inflammatory bowel disease?, Eur J Intern Med 11, 197-203]. Consequently, the primary objective of the clinical management of IBD is to induce and maintain remission of disease activity and reduce complications. Current therapy of IBD usually involves the administration of anti-inflammatory or immunosuppressive agents, such as aminosalicylates, sulfasalazine, corticosteroids, 6-mercaptopurine/azathioprine, anti-inflammatory anti-cytokine antibodies, and the like. For example, U.S. Pat. No. 6,395,273 to Kink et al., discloses polyclonal anti-TNF antibodies for reduction of pro-inflammatory TNF levels in IBD. U.S. Pat. No. 6,482,409 to Lobb et al., discloses the use of VCAM-1/IgG fusion proteins and antibodies recognizing the integrin VLA4 (very late antigen-4) in the treatment of IBD. U.S. Pat. No. 6,479,465 to Strober et al., discloses the use of anti-STAT-4 (Signal Transducers and Activators of Transcription, which regulate Th1 and Th2 differentiation) antisense oligonucleotides for treatment and prevention of the inflammatory response in IBD. U.S. Pat. No. 6,540,993 to Warne, et al., discloses the use of topical formulations of the anti-inflammatory cytokine interleukin-11 for treating IBD and other inflammatory gastrointestinal diseases. U.S. Pat. No. 6,558,661 to Ashkenazi et al., discloses the use of inhibitors of the pro-inflammatory cytokine IFN-γ for attenuation of inflammatory processes in IBD.

In another, non-pharmaceutical approach, U.S. Pat. No. 6,491,618 to Ganz, discloses an apparatus and method for intracorporeal ultra violet light irradiation of the stomach or colon, for treatment of gastrointestinal microbial overgrowth characteristic of IBD.

When anti-inflammatory/immunosuppressive therapies fail, colectomies are a radical but common procedure: about 30% of Crohn's disease patients will require surgery within the first year after diagnosis. In the subsequent years, the rate is about 5% per year. Unfortunately, Crohn's disease is characterized by a high rate of recurrence; about 5% of patients need a second surgery each year after initial surgery. In ulcerative colitis, a further reason for resorting to surgery exists due to the much increased risk of ulcerative colitis patients for developing colorectal cancer, starting 10-15 years after the diagnosis of ulcerative colitis. Presumably, this is due to the recurrent cycles of injury to the epithelium, followed by regrowth, increasing the risk of cancerous transformation of the affected mucosal cells. Accordingly, colostomy is often recommended as prophylaxis against the development of cancer in ulcerative colitis patients.

Despite the numerous suggested therapies, when results of the many studies of IBD treatment are surveyed, the overall conclusion is that present methods of treatment can provide only partial relief at best [Beattie et al., (1994). Polymeric nutrition as the primary therapy in children with small bowel Crohn's disease, Aliment Pharmacol Ther 8, 609-15]. Furthermore, severe side effects are associated with the drugs commonly prescribed for IBD, including nausea, dizziness, changes in blood chemistry (including anemia and leukopenia), skin rashes and drug dependence. The surgical treatments commonly resorted to are radical procedures that often profoundly alter everyday life for the patient. Accordingly, there is a great need for treatments for IBD that are effective yet less severe in their side effects, less invasive and would improve quality of life for IBD patients.

Nutritional Therapy and Selenium:

Nutritional therapy is commonly recommended for a great variety of diseases, usually based on perceived dietary deficiencies, intent to alter specific metabolic processes, or both. Nutrition therapy has the advantage of incorporating elements normally found in foods and nutritional materials, being potentially better tolerated than drugs, and being plentifuly available in natural form, thus being usually more inexpensive than manufactured drugs.

Selenium is a trace mineral having nutritional importance as a cofactor of a number of metalloenzymes, most notable enzymes active in scavenging of free radicals and other anti-oxidant functions, e.g., glutathione peroxidase and thioredoxin reductase [Arner, E. S., and Holmgren, A. (2000). Physiological functions of thioredoxin and thioredoxin reductase, Eur J Biochem 267, 6102-9; Brigelius-Flohe, R, et al (2000). Overexpression of PHGPx inhibits hydroperoxide-induced oxidation, NFkappaB activation and apoptosis and affects oxLDL-mediated proliferation of rabbit aortic smooth muscle cells, Atherosclerosis 152, 307-16]. Since low levels of nutritional selenium intake, and reduced levels of serum and tissue selenium have been correlated with a number of inflammatory and cancerous conditions (see, for example, Rannem et al, Am J Clin Nutr 1992;56:933-37), selenium supplementation has been included in a number of therapeutic nutritional compositions (Rayman and Rayman Proc Nutr Soc 2002;61:203-15).

For example, U.S. Pat. No. 6,524,619 to Pearson et al. discloses clinically useful formulations comprising selenium (0.04-1.0 mg/person/day, as an antioxidant) in addition to other trace minerals, folate, riboflavin, micronutrients and supplements for treatment of hearing loss, deafness and tinnitus by reducing free radical levels, and enhancing vascular and endothelial health. U.S. Pat. No. 5,156,852 to La Haye et al. teaches that a composition containing vitamin E and low doses (80-100 μg/day) of selenium, among other cofactors for free radical scavenging metalloenzymes, is useful in treating macular degeneration and other eye diseases. U.S. Pat. No. 6,444,221 to Shapiro discloses the use of carbonyl trapping agents such as primary amine derivatives of benzoic acid, in combination with anti-oxidant and anti-inflammatory coagents such as selenium, for sequestering products of lipid peroxidation. U.S. Pat. No. 5,405,613 to Rowland teaches natural vitamin and mineral compositions that include 20-50 μg selenium and bioflavonoids for restoring energetic balance or intensity. U.S. Pat. No. 5,639,482 to Crary teaches the administration of glutathione in combination with vitamin E and 250 or 1000 μg/day of selenium compounds to prevent capillary leakage and bleeding in diabetics, especially for treatment of diabetic retinopathy. U.S. Pat. Nos. 6,242,010 and 6,303,651 to Hersch teach topical preparations comprising glutathione and a selenium source as antioxidants for treatment of ano-rectal wounds.

Recently, a number of blinded, prospective clinical trials have investigated the efficacy of nutritional anti-oxidant supplementation, in the form of combined Vitamin E and selenium administration, for prevention of cancer and neurological degenerative disease. The SELECT study (Selenium and Vitamin E Cancer Prevention Trial, National Cancer Institute and SouthWest Oncology Group) and PREADVISE study (Prevention of Alzheimer's Disease by Vitamin E and Selenium, University of Kentucky) have investigated the effects of supplementation of Vitamin E (400 IU/day) and selenium (200 μg/day) in large population samples around North America (see www.crab.org/select). The data accumulated from these and other similar studies has indicated that Vitamin E and low doses of selenium supplementation can reduce the incidence of total and prostate cancer, but not skin, lung or colorectal cancer (Duffield-Lillico et al, Canc. Epidem. Biomark Rev 2002;11:630-39; Duffield-Lillico et al, BJU Int. 2003;91:608-12; Klein et al World J Urol 2003;21:21-27; Clark et al, JAMA 1996;276:1957-63). It will be noted, however, that in these studies, the Vitamin E and selenium supplementation was most effective in groups characterized by low baseline levels of selenium and cancer markers (such as PSA for prostate cancer), and high incidence of smoking. Similarly, results of animal studies on selenium supplementation have indicated a role for selenium in prevention of tumors and metastatic transformation (Thiunavuckarasu et al, Pharmacol Res, 2003;48:167-73; Sivaram et al Comp Biochem Physiol C Toxicol Pharmacol 2003;134:397401; Yan et al, Anticancer Res 1999;19:1337-42). As a result of such studies, antioxidant vitamin (A,C,D and E) and mineral cofactor (including selenium) have been recommended to become a part of chemopreventive strategy for prostate cancer (Ansari et al., Int. Urol Nephrol 2002;34:207-14).

Some of the proposed nutritional treatments for IBD stress the enhanced requirement for antioxidants and minerals such as selenium in a disease characterized by malabsorption, extensive tissue damage and varying degrees of malnutrition. Indeed, herbal remedies used by patients for treatment of inflammatory bowel disease include slippery elm, fenugreek, devil's claw, Mexican yam, tormentil and wei tong ning, all having anti-oxidant activity (Langmead L et al, Aliment Pharmac Ther 2002;16:197-205). Thus, selenium is also a common component of numerous nutritional compositions for enteral nutrition in metabolically stressed patients having compromised nutrition or malnutrition, resulting from gastrointestinal conditions such as food allergies, sick gut disease, IBD, etc. Such compositions typically include low doses of selenium compounds to supply RDA-level (about 20-100 μg/day) selenium supplementation (see, for example, U.S. Pat. No. 5,661,123 to Stalker et al, U.S. Pat. No. 5,922,766 to Acosta et al, U.S. Pat. No. 6,051,260 to Liska et al., U.S. Pat. No. 6,585,998 to Cartwright et al, and U.S. Pat. No. 6,200,950 to Mark et al., all incorporated herein by reference as if fully incorporated herein).

It has been reported that selenium levels are lower in patients with IBD [Geerling B J, et al., Eur J Clin Nut 2000;54:514-21; Rannem et al Scand J Gastroenter 1998;33:1057-61; Ringstad et al., (1993). Serum selenium, copper, and zinc concentrations in Crohn's disease and ulcerative colitis, Scand J Gastroenterol 28, 605-8]. However, it remains unclear whether this deficiency is causative of, or resultant from, the gastrointestinal inflammation associated with IBD (Ishida T et al. Intern Med. 203;42:154-57; Phylactos et al, Acta Paediat. 2001;90:883-88; Reimund J M et al Clin Nutr 2000;19:43-48). Thus, restorative selenium supplementation, for anti-oxidant effect, is included in a number of proposed treatment regimens for IBD. For example, U.S. Pat. No. 5,444,044 to Garleb et al; and U.S. Pat. Nos. 5,925,314, 6,468,987 and 5,780,451 to DeMichele et al, disclose a nutritional product for treating ulcerative colitis or inflammation of the colon which contains in combination an oil blend containing fatty (eicosapentaenoic and/or docosahexaenoic) acids and a source of indigestible carbohydrate able to be metabolized to short chain fatty acids by gastrointestinal flora. Selenium (in low, sub-RDA doses), was also added, along with Vitamin A, Vitamin E, and other nutrients which act as antioxidants to reduce levels of reactive oxygen species associated with gastrointestinal inflammation. The disclosed nutritional products are designed to provide nutritional replenishment to counterbalance the deficiency and malnutrition common in dysfunctional digestion in IBD. Similarly, U.S. Pat. Nos. 6,420,342 and 6,548,483 to Hageman et al., disclose nutritional compositions containing ribose and folic acid, in combination with niacin, histidine, vitamin B6 and other components, including selenium (in low doses for antioxidant effects), for treatment and prevention of various diseases, among them inflammatory gastrointestinal conditions. However, there is no strong evidence thus far demonstrating significant improvement in any clinical trials using anti-oxidants as therapeutic agents in IBD (for a comprehensive review, see Ling, et al Current Opin Clin Nutr and Metab Care 2000;3:339-44).

Selenium Attenuation of Immune Overstimulation:

Little information is available on the biological activity of selenium or on its function in its enzyme-free form; most studies published to date have investigated the activity of Selenium while incorporated into selenoproteins (Arner, E. S., et al, Eur J Biochem, 2000; 267; 6102-6109, Brigelius-Flohe, R., et al, Atherosclerosis 2000; 152: 307-316, Schulz, H. U., et al, Hepatogastroenterology 1999;46: 2736-275, Molina, H., et al, Mech Ageing Dev, 1997; 97:1-7). Administration of selenium has been suggested in diseases such as sepsis (for a review, see Gartner, et al, Biofactors, 2001;14:199-204), although the precise underlying mechanism is not known.

The pathophysiology of certain inflammatory diseases or conditions is associated with influx of macrophages to the site(s) of inflammation, and subsequent pathological changes in the morphology and function of the affected organ. Such macrophage-mediated conditions include nervous system diseases such as Guillian-Barre syndrome, renal disease such as glomerulonephritis, coronary disease such as atherosclerosis, and generalized macrophage infiltration as in histiocytosis.

Crescentic glomerulonephritis is a disease of broad etiology. The initiating event is the development of a physical disruption in the GBM. The lesions are mediated by processes involving macrophages and cell-mediated immunity, which lead to development of crescents.

The major participants in crescent formation are coagulation proteins, macrophages, T cells, fibroblasts, and parietal epithelial cells. Activated macrophages contribute to the crescents by proliferating and releasing procoagulant tissue factor, interleukin-1 (IL-1) and tumor necrosis factor (TNF). T cells are not prominent components, but they play an important role in glomerular injury by antigen recognition and macrophage recruitment. The reversibility of crescents corresponds with relative predominance of cellular components. 73% of treated patients either die or require dialysis, and the current therapy, admittedly generally unsuccessful, is immunosuppression.

The mechanisms of atherogenesis remain uncertain. Endothelial injury causes vascular inflammation and a fibroproliferative response ensues. Circulating monocytes infiltrate the intima of the vessel wall, and these tissue macrophages act as scavenger cells, taking up LDL cholesterol and forming the characteristic foam cell. These activated macrophages produce numerous factors that are injurious to the endothelium. Elevated serum levels of LDL cholesterol overwhelm the antioxidant properties of the healthy endothelium and result in abnormal endothelial metabolism. Oxidized LDL also activates inflammatory processes at the level of gene transcription by up-regulation of nuclear factor kappa-B, expression of adhesion molecules, and recruitment of monocytes/macrophages.

The earliest pathologic lesion of atherosclerosis is the fatty streak, the result of focal accumulation of serum lipoproteins within the intima of the vessel wall. Microscopy reveals lipid-laden macrophages, T lymphocytes, and smooth muscle cells in varying proportions.

Denudation of the overlying endothelium or rupture of the protective fibrous cap may result in exposure of the thrombogenic contents of the core of the plaque to the circulating blood. Activated macrophages produce matrix metalloproteinases that degrade collagen. These mechanisms explain the predisposition to plaque rupture and highlight the role of inflammation in the genesis of the complications of the fibrous atheromatous plaque.

Histiocytosis encompasses a group of diverse disorders that have in common, as a primary event, the accumulation and infiltration of monocytes, macrophages, and dendritic cells in the affected tissues, excluding diseases in which infiltration is in response to a primary pathology. The spectrum of clinical presentation in this group of disorders varies greatly, ranging from mild to life threatening. Although nearly a century has passed since the recognition of histiocytic disorders, their pathophysiology remains an enigma, and currently the treatment is nonspecific in character.

Guillian-Barre syndrome (AIDP) is believed to be caused by an immunologic attack that is directed against myelin components, resulting in a demyelinating polyneuropathy. Early inflammatory lesions consist of a lymphocytic infiltrate that is adjacent to segmental demyelination, with macrophages prominent several days later.

With electron microscopy, macrophages are observed stripping off the myelin sheath. Antimyelin antibodies and complement direct macrophages to Schwann cells by opsonization. The changes are observed in nerve roots, peripheral nerves, and cranial nerves. In acute motor axonal neuropathy (AMAN, an AIDP variant), deposited complement is found at the nodes of Ranvier, while myelin often is left undamaged.

Damage to the myelin sheath leads to segmental demyelination. This results in decreased nerve conduction velocity, conduction block and occasionally axonal degeneration which results in wallerian degeneration Commonly, AIDP refers to the more common demyelinating form unless otherwise specified.

U.S. Pat. No. 5,939,394 to Fleming et al and U.S. Pat. No. 6,350,467 to Demopoulos et al teach the use of Selenium in compositions with glutathione, magnesium, and other antioxidants for treatment of allergic, autoimmune and inflammatory conditions. U.S. Pat. No. 5,973,009 to Tailhan-Lomont et al teaches the administration of cyclic diselenide and selenosulfide compounds for overproduction of peroxides. However, none of the prior art documents teach short-term high dose dietary selenium supplementation for inflammatory conditions.

Excessive and continuous cytokine production in response to bacterial lipopolysaccharides (LPS) or superantigens is a hallmark of the systemic inflammatory response (SIRS), which can be life-threatening. Selenium administration; along with other antioxidants to selenium depleted SIRS patients dramatically improves prognosis, indicating an inhibitory effect on an over-stimulated immune system.

A mechanism by which selenium might modulate the functioning of the immune system could involve induction of necrotic cell death or apoptosis of macrophages, and a reduction in peroxide production. Modulating these parameters in macrophages may be a key event in modulating the inflammatory response, particularly in macrophage-mediated inflammatory conditions.

There is thus a widely recognized need for, and it would be highly advantageous to have, a method of treating inflammatory disease, and IBD in particular, with effective, short-term high dose dietary selenium supplementation devoid of the above limitations.

Mitochondrial Dysfunction and Disease

Mitochondria contain their own DNA genome. These organellar genomes encode a fraction of the gene products required for organellar function, the remainder of such gene products being encoded by the nuclear genome. Relatively little is known about the mechanisms by which mitochondrial gene products, which may be encoded by nuclear sequences or sequences found in the organellar genomes, are coordinately regulated (Surpin and Chory, Essays Biochem. 1997,32:113-125).

The organelle known as the mitochondrion is the main energy source in cells of higher organisms. Mitochondria provide direct and indirect biochemical regulation of a wide array of cellular respiratory, oxidative and metabolic processes. These include electron transport chain (ETC) activity, which drives oxidative phosphorylation to produce metabolic energy in the form of adenosine triphosphate (ATP), and which also underlies a central mitochondrial role in intracellular calcium homeostasis.

In addition to their role in energy production in growing cells, mitochondria (or, at least, mitochondrial components) participate in programmed cell death (PCD), also known as apoptosis (Newmeyer et al., Cell 1994, 79:353-364; Liu et al., Cell 1996, 86:147-157). Apoptosis is apparently required for normal development of the nervous system, and for proper functioning of the immune system. Moreover, some disease states are thought to be associated with either insufficient or excessive levels of apoptosis (e.g., cancer and autoimmune diseases in the first instance, and stroke damage and neurodegeneration in Alzheimer's disease in the latter case). For general reviews of apoptosis, and the role of mitochondria therein, see, e.g., Green and Reed (Science, 1998, 281:1309-1312), Green (Cell, 1998, 94:695-698) and Kromer (Nature Medicine, 1997,3:614-620). Altered or defective mitochondrial activity, including but not limited to failure at any step of the ETC, may result in the generation of highly reactive free radicals that have the potential of damaging cells and tissues. These free radicals may include reactive oxygen species (ROS) such as superoxide, peroxynitrite and hydroxyl radicals, and potentially other reactive species that may be toxic to cells. For example, oxygen free radical induced lipid peroxidation is a well established pathogenetic mechanism in central nervous system (CNS) injury such as that found in a number of degenerative diseases, and in ischemia (i.e., stroke).

In addition to free radical mediated tissue damage, there are at least two deleterious consequences of exposure to reactive free radicals arising from mitochondrial dysfunction that adversely impact the mitochondria themselves. First, free radical mediated damage may inactivate one or more of the myriad proteins of the ETC. Second, free radical mediated damage may result in catastrophic mitochondrial collapse that has been termed “permeability transition” (PT) or “mitochondrial permeability transition” (MPT). According to generally accepted theories of mitochondrial function, proper ETC respiratory activity requires maintenance of an electrochemical potential (Δψ) in the inner mitochondrial membrane by a coupled chemiosmotic mechanism, as described herein. Free radical oxidative activity may dissipate this membrane potential, thereby preventing ATP biosynthesis and halting the production of a vital biochemical energy source. In addition, mitochondrial proteins such as cytochrome c and “apoptosis inducing factor” may leak out of the mitochondria after permeability transition and may induce the genetically programmed cell suicide sequence known as apoptosis or programmed cell death (PCD).

Altered mitochondrial function characteristic of the mitochondria associated diseases may also be related to loss of mitochondrial membrane electrochemical potential by mechanisms other than free radical oxidation, and permeability transition may result from direct or indirect effects of mitochondrial genes, gene products or related downstream mediator molecules and/or extramitochondrial genes, gene products or related downstream mediators, or from other known or unknown causes. Loss of mitochondrial potential therefore may be a critical event in the progression of diseases associated with altered mitochondrial function, including degenerative diseases. Mitochondrial defects, which may include defects related to the discrete mitochondrial genome that resides in mitochondrial DNA and/or to the extramitochondrial genome, which includes nuclear chromosomal DNA and other extramitochondrial DNA, may contribute significantly to the pathogenesis of diseases associated with altered mitochondrial function. A number of degenerative, hyperproliferative and other types of diseases are thought to be caused by, or to be associated with, alterations (e.g., statistically significant increases or decreases) in mitochondrial function. These include, for example, Alzheimer's Disease, Parkinson's Disease, Huntington's disease, diabetes mellitus, and hyperproliferative disorders, such as cancer, tumors and psoriasis. Additional diseases with which altered mitochondrial function or mitochondrial dysfunction has been associated include amyotrophic lateral sclerosis (ALS), Friedreich's ataxia, colon cancer, and exercise intolerance.

The majority of gene products required for mitochondrial respiratory function are encoded in the nuclear genome. One approach to understanding nucleo-mitochondrial (or mitonuclear) interactions in mammalian cells has been the identification of nuclear transcription factors that regulate the expression of such gene products. For example, using this approach two transcription factors known as nuclear respiratory factors-1 and -2 (NRF-1 and NRF-2) have been purified, and nucleic acid sequences encoding NRF-1 and NRF-2 have been molecularly cloned. The DNA binding and transcriptional specificities of these proteins have implicated them in the expression of many respiratory subunits along with key components of, inter alia, mitochondrial transcription, replication and heme biosynthetic mechanisms.

Another molecule critical in mitochondrial biogenesis and function is the mitochondrial transcription factor A (mtTFA or Tfam). A key regulator of transcription and replication of mitochondrial DNA, mtTFA has also been shown to be essential to the integrity of mitochondrial DNA, which lacks histones and nucleosome conformations (Kanki et al., Ann NY Acad Sci 2004;104:61-68).

mtTFA expression, as well as the expression of a number of genes associated with anti-oxidant-related genes such as glutamate cysteine-ligase (GCL) and NAD(P)H:quinone oxidoreductase 1 and cytochrome oxidase, is regulated by NRF-1 (Bea et al. Circ Res 2003, 92:386-93, Leung et al JBC, 2003, 278:48021-29). Aberrant expression of NRF-1 and/or mtTFA has been observed in a number of pathological conditions, such as insulin resistance, type 2 diabetes mellitus and cardiomyopathy (Patt et al, PNAS 2003;100:8466-71, Siciliano et al Neurol Science 2000;21:S985-97 and Wallace et al, Methods Mol Biol 2002;197:3-54). Intentional alteration in NRF-1 and/or mtTFA levels, via genetic manipulation, leads to early embryonic lethality, severe oxidative stress, diabetes, neonatal cardiomyopathy and neonatal sensitivity to TNF, (Leung et al, JBC 2003;278:48021-29 and Wallace D C, Method Mol Biol 2002; 197:3-54). Recently, it has been demonstrated that replenishing mtTFA in deficient rat liver directly increases mitochondrial RNA transcription (Garstka et al. Nucl Acids Res 2003;104:61-68).

The present inventors have shown that high dose dietary selenium supplementation protects against inflammatory bowel syndrome and mitochondrial dysfunction in an animal model of the inflammatory disease [Tirosh et al., Clin Nutr 2003; 22(S1):S34]. In view of the importance of mitochondrial function in conditions of disease and health, there is thus a widely recognized need for, and it would be highly advantageous to have, a novel method of treating or preventing mitochondrial dysfunction, with effective, short-term high dose dietary selenium supplementation devoid of the above limitations.

### SUMMARY OF THE INVENTION

According to the present invention there is provided a method for treating a subject having an inflammatory disease or condition comprising administering to the subject selenium in a daily amount of about 0.1 μmol to about 100 μmol per kg body weight, for at least one day, thereby treating the subject for the inflammatory disease or condition.

According to further features in preferred embodiments of the invention described below, administering is for not more than 21 days, or not for more than 12 months.

According to yet further features in preferred embodiments of the invention described below, the daily amount is about 2 μmol to about 50 μmol, per kg body weight, preferably about 10 μmol to about 20 μmol per kg body weight.

According to still further features in preferred embodiments of the invention described below, the selenium is administered as a constituent of a selenium source. The selenium source can be selected from the group consisting of an inorganic selenium compound and an organoselenium compound.

According to further features in preferred embodiments of the invention described below, the inorganic selenium compound is selected from the group consisting of sodium selenite, sodium selenide, sodium selenate, selenic acid, selenium sulfide, selenium oxychloride, selenium dioxide, hydrogen selenide and selenious acid.

According to still further features in preferred embodiments of the invention described below, the organoselenium compound is selected from the group consisting of dimethyl selenium, trimethyl selenium, selenomethionine, selenocysteine, seleno-methyl selenocysteine, selenocystathionine, selenotaurine, selenodiglutathione, allyl selenocysteine, propyl selenocysteine, selenoethionine and selenocystamine.

According to yet further features in preferred embodiments of the invention described below, the inflammatory disease is selected from the group consisting of hypersensitivity, an autoimmune disease, an infectious disease, graft rejection, an allergic disease, an inflammatory musculo-skeletal disease, a gut-related inflammatory disease, a neurological inflammatory disease, an inflammatory cardiovascular disease, an injury, an idiopathic inflammatory disease and an inflammation of unknown etiology.

According to further features in preferred embodiments of the invention described below, the gut-related inflammatory disease is selected from the group consisting of inflammatory bowel disease, mucositis, necrotizing enterocolitis, aphthous stomatitis, pharyngitis, esophagitis, peptic ulcers, gingivitis and periodontitis.

According to still further features in preferred embodiments of the invention described below, inflammatory bowel disease is selected from the group consisting of Crohn's disease, ulcerative colitis, indeterminate colitis, and infectious colitis.

According to yet further features in preferred embodiments of the invention described below, the hypersensitivity is selected from the group consisting of hypersensitivity is selected from the group consisting of Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and delayed type hypersensitivity.

According to further features in preferred embodiments of the invention described below, the delayed type hypersensitivity is selected from the group consisting of contact dermatitis and drug eruption.

According to still further features in preferred embodiments of the invention described below, the inflammatory cardiovascular disease is selected from the group consisting of occlusive disease, atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity in Chagas' is disease and anti-helper T lymphocyte autoimmunity.

According to yet further features in preferred embodiments of the invention described below, the neurological inflammatory disease is selected from the group consisting of neurodegenerative disease, multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis and stiff-man syndrome.

According to further features in preferred embodiments of the invention described below, the infectious disease is selected from the group consisting of chronic infectious disease, subacute infectious disease, acute infectious disease, viral disease, bacterial disease, protozoan disease, parasitic disease, fungal disease, mycoplasma disease and prion disease.

According to still further features in preferred embodiments of the invention described below, the graft rejection is chronic graft rejection, subacute graft rejection, hyperacute graft rejection, acute graft rejection and/or graft versus host disease.

According to yet further features in preferred embodiments of the invention described below, the allergic disease is selected from the group consisting of asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

According to further features in preferred embodiments of the invention described below, the inflammatory musculo-skeletal disease is selected from the group consisting of autoimmune myositis, primary Sjogren's syndrome, smooth muscle autoimmune disease, rheumatoid arthritis, ankylosing spondylitis, muscle inflammation, myositis, a tendon inflammation, tendinitis, a ligament inflammation, a cartilage inflammation, a joint inflammation, a synovial inflammation, carpal tunnel syndrome and a bone inflammation.

According to still further features in preferred embodiments of the invention described below, the autoimmune disease is selected from the group consisting of Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome, autoimmune ear disease, autoimmune disease of the inner ear, autoimmune interstitial nephritis, autoimmune bullous skin disease, pemphigus vulgaris, bullous pemphigoid and pemphigus foliaceus.

According to yet further features in preferred embodiments of the invention described below, administering is carried out twice daily.

According to yet another aspect of the present invention there is provided a method for treating a subject having a macrophage-mediated inflammatory disease or condition comprising administering to the subject selenium in a daily amount of about 0.1 μmol to about 100 μmol per kg body weight, for at least one day, thereby treating the subject for the macrophage-mediated inflammatory disease or condition.

According to further features in preferred embodiments of the invention described below, the macrophage-mediated inflammatory disease is selected from the group consisting of atherosclerosis, glomerulonephritis, histiocytosis and Guillain-Barre syndrome.

According to yet another aspect of the present invention, there is provided a method for treating a subject having a disease or condition associated with altered mitochondrial function comprising administering to the subject selenium in a daily amount of about 0.1 μmol to about 100 μmol per kg body weight, for at least one day, thereby treating the subject for the disease or condition.

According to further features in preferred embodiments of the invention described below, wherein said disease or condition associated with altered mitochondrial function is selected from the group consisting of Alzheimer's Disease, Parkinson's Disease, Huntington's disease, progressive supranuclear palsy, diabetes mellitus, hyperproliferative disorders such as cancer, tumors and psoriasis, amyotrophic lateral sclerosis (ALS), Friedreich's ataxia, colon cancer, stroke, exercise intolerance and cardiac myopathy.

According to another aspect of the present invention, there is provided a selenium solid oral dosage form unit comprising as an active ingredient from about 0.005 to about 10 mmol of selenium in a volume of less than 1 cm³ and a pharmaceutically acceptable carrier.

According to further features in preferred embodiments of the invention described below, the solid oral dosage form unit further comprises a pharmaceutically acceptable excipient selected from the group consisting of carboxymethylcellulose, microcrystalline cellulose, starch, and modified starch.

According to yet further features in preferred embodiments of the invention described below, the selenium dosage form unit is selected from the group consisting of a tablet, caplet, capsule, granules, beads, particles and pellets.

According to still another aspect of the present invention there is provided a method for downregulating an activity of an inflammatory immune cell, the method comprising exposing the cell to a concentration of selenium sufficient to downregulate an inflammatory process in the cell, thereby downregulating an activity of the inflammatory immune cell.

According to further features in preferred embodiments of the invention described below, said concentration of selenium is about 0.1 μM to about 1000 μM selenium, more preferably about 0.5 μM to about 50 μM.

According to yet further features in preferred embodiments of the invention described below, exposing the cell is performed in vivo or in vitro.

According to still further features in preferred embodiments of the invention described below, said inflammatory process comprises production of reactive oxygen species production and said downregulation is via reduced expression of mitochondrial proteins such as MnSOD and UCP2.

According to further features in preferred embodiments of the invention described below said inflammatory immune cell is selected from the group consisting of T-lymphocytes, dendritic cells, eosinophils, macrophages, granulocytes, monocytes and macrophages.

According to another aspect of the present invention there is provided article of manufacture comprising a packaging material and at least one selenium unit dosage, the selenium unit dosage comprising a pharmaceutical composition comprising from about 0.005 to about 10 mmol selenium and a pharmaceutically acceptable carrier in each single unit dosage. The packaging material comprises a label or package insert indicating that the pharmaceutical composition is for treating and/or preventing an inflammatory disease or condition.

According to further features in preferred embodiments of the invention described below, the selenium unit dosage comprises about 0.05 mmol to about 5 mmol selenium, preferably about 0.5 mmol to about 2.5 mmol selenium.

According to still further features in preferred embodiments of the invention described below, the pharmaceutical composition comprises selenium as a constituent of a selenium source. The selenium source can be an inorganic selenium compound and an organoselenium compound.

According to yet further features in preferred embodiments of the invention described below, the inorganic selenium compound is selected from the group consisting of sodium selenite, sodium selenide, sodium selenate, selenic acid, selenium sulfide, selenium oxychloride, selenium dioxide, hydrogen selenide and selenious acid.

According to further features in preferred embodiments of the invention described below, the organoselenium compound is selected from the group consisting of dimethyl selenium, trimethyl selenium, selenomathionine, selenocysteine, seleno-methyl selenocysteine, selenocystathionine, selenotaurine, selenodiglutathione, allyl selenocysteine, propyl selenocysteine, selenoethionine and selenocystamine.

According to still further features in preferred embodiments of the invention described below, the composition further comprises a pharmaceutically acceptable excipient selected from the group consisting of carboxymethylcellulose, microcrystalline cellulose, starch, and modified starch.

According to yet further features in preferred embodiments of the invention described below, the selenium unit dosage is designed for oral administration.

According to further features in preferred embodiments of the invention described below, the selenium unit dosage is selected from the group consisting of a tablet, a caplet, and a capsule.

According to still further features in preferred embodiments of the invention described below, the composition is in the form of a liquid dosage form.

The present invention successfully addresses the shortcomings of the presently known configurations by providing a method short-term, high dose dietary selenium supplementation for treating a subject having an inflammatory disease. The method of the present invention is based on the finding that short term dietary selenium supplementation in great excess of the conventional RDA can inhibit inflammation and inflammatory processes associated with inflammatory bowel disease (IBD). An article of manufacture comprising a pharmaceutical composition providing a selenium unit dosage suitable for the method of the present invention is also provided.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph showing enhancement of levels of colon tissue selenium by high dose selenium dietary supplementation. Samples of colon tissue of rats receiving three weeks of high dose selenium dietary supplementation of 16 ppm sodium selenite in the drinking water (columns B and D) or no selenium dietary supplementation (columns A and C) were prepared by microwave assisted digestion, and analyzed for selenium by ICP-atomic emission spectrometry. Tissue selenium levels were measured in colon tissue from healthy rats (columns A and B), and from rats 24 hours after induction of colitis with hapten (TNBS, 100 mg/ml) intra-anally (columns C and D). Note the enhanced levels of colon tissue selenium in tissue from the rats receiving high dose selenium dietary supplementation (columns B and D), compared to the untreated controls (A and C);

FIGS. 2A-2D is a series of photographs illustrating the protective effect of high dose selenium dietary supplementation on gross pathological parameters of colon tissue. Resected colons from rats receiving three weeks of high dose selenium dietary supplementation of 16 ppm sodium selenite in the drinking water (2B and 2D) or no selenium dietary supplementation (2A and 2C) were photographed. Extent of necrosis and inflammation were observed in colon tissue from healthy rats (2A and 2B), and from rats 24 hours after induction of colitis with hapten (TNBS, 100 mg/ml) intra-anally (2C and 2D). Note the mild necrosis in the colon of hapten-induced (colitis) rats receiving high dose selenium dietary supplementation (2C) compared with the extensive and severe necrosis apparent in the tissue from PBS-treated controls (2D);

FIGS. 2E-2G are a series of representative photographs and histology sections illustrating the correlation between the protective effect of high dose dietary selenium supplementation on gross pathological and microscopic-histological parameters of colon tissue. Resected colons from healthy (untreated) rats (2E), rats 24 hours after hapten (TNBS)-induced colitis (2F), as above, or from colitis-induced rats receiving selenium supplementation, as above (2G), are compared for gross pathology (lower panel), and histological parameters (upper panel). Note the mild proprial edema and erosion of the mucosal epithelium with dietary selenium supplementation (2G), compared with the predominance of mucosal necrosis, hemorrhaging and neutrophilic infiltration in the tissue from the hapten-induced, untreated animals (2F).

FIG. 3 is a graph showing reduction of inflammation-associated myeloperoxidase (MPO) activity in colon tissue by high dose selenium dietary supplementation. Samples of colon tissue of rats receiving three weeks of high dose selenium dietary supplementation of 16 ppm sodium selenite in the drinking water (columns B and D Se suppl+) or no selenium dietary supplementation (columns A and C Se suppl−) were homogenized, frozen and thawed, and analyzed spectrophotometrically for MPO activity, indicative of infiltration of neutrophils following inflammation. Tissue MPO levels (expressed in Units/gram) were measured in colon tissue from healthy rats (columns A and B), and from rats 24 hours after induction of colitis with hapten (TNBS, 100 mg/ml) intra-anally (columns C and D). Note the extensive protection (>80%) against TNBS-induced inflammation in tissue from the rats receiving high dose selenium dietary supplementation (column D);

FIG. 4 is a graph illustrating the contribution of mitochondrial respiration to respiration in colon tissue. Respiratory activity was analyzed by measuring oxygen consumption in fresh colon tissue by polarographic measurement using an oxygen electrode. Inhibition of the Electron Transport Chain (ETC) with the mitochondrial-specific metabolic inhibitor rotenone (+rotenone) significantly reduced tissue respiration, compared with untreated controls (−rotenone), indicated that most of the respiratory activity in colon tissue is directly associated with mitochondrial respiration;

FIG. 5 is a graph showing protection against inflammation-associated suppression of respiration in colon tissue by high dose selenium dietary supplementation. Samples of colon tissue of rats receiving three weeks of high dose selenium dietary supplementation of 16 ppm sodium selenite in the drinking water (columns B and D, Se suppl+) or no selenium dietary supplementation (columns A and C, Se suppl−) were incubated at ambient temperature with PBS supplemented with 5 mM glucose, and oxygen consumption in colon tissue was measured by polarographic measurement using an oxygen electrode. Oxygen consumption levels were measured in colon tissue from healthy rats (columns A and B), and from rats 24 hours after induction of colitis with hapten (TNBS, 100 mg/ml) intra-anally (columns C and D). Note the significant protection against TNBS-induced respiratory arrest provided by high dose dietary selenium supplementation (column C compared to column D);

FIG. 6 shows a PCR analysis of the protective effects of high dose selenium dietary supplementation on mitochondrial DNA loss in colon inflammation. The content of mitochondrial DNA in colon tissue samples from healthy animals (con) or animals with hapten-induced colitis (TNBS), either receiving high dose selenium dietary supplementation (Se suppl+) of 16 ppm sodium selenite in the drinking water or no selenium dietary supplementation (Se suppl−), was determined using specific primers for PCR amplification of the D-loop region of mitochondrial DNA. Mitochondrial DNA was assayed in colon tissue from healthy rats (colitis−), or from rats 24 hours after induction of colitis with hapten (TNBS, 100 mg/ml) intra-anally. Graph showing densitometry units (lower panel) corresponds to the intensity of mtDNA bands in the representative EtBr stained gel shown in the upper panel. Values are mean±SD, n=5 (TNBS). Note that high dose selenium dietary supplementation (Se-TNBS, lane 4) effectively protects against inflammation-induced loss of mitochondrial DNA (see TNBS, lane 3);

FIGS. 7A-7B are a PCR analysis, and Western blot illustrating the specific inhibition of inflammation-induced alterations in mRNA levels and three dimensional configuration of cytochrome c by high dose selenium dietary supplementation. FIG. 7A is a gel of the products of RT-PCR analysis of cytochrome c and housekeeping genes expression in pooled RNA from 6 animals receiving three weeks of high dose selenium dietary supplementation of 16 ppm sodium selenite in the drinking water (lanes 2 and 4, Se suppl+) or no selenium dietary supplementation (lanes 1 and 3, Se suppl−). Abundance of cytochrome c, 18S and GADPH transcripts in colon tissue from healthy rats (lanes 1 and 2, TNBS−), and from rats 24 hours after induction of colitis with hapten (TNBS, 100 mg/ml) intra-anally (lanes 3 and 4, TNBS+) was detected using specific primers, and gel-separated PCR products visualized by EtBr staining. Note the specificity of the protective effect of high dose dietary selenium supplementation on cytochrome c expression, while the housekeeping genes 18S RNA and GAPDH remain unaffected. FIG. 7B is a Western blot of colon tissue illustrating the same protective effect at the protein level. Samples of colon tissue of rats receiving three weeks of high dose selenium dietary supplementation of 16 ppm sodium selenite in the drinking water (lanes 2 and 4, Se suppl+) or no selenium dietary supplementation (lanes 1 and 3, Se suppl−) were subjected to protein denaturation (boiling, SDS-PAGE), and separated proteins transferred to nitrocellulose membrane by blotting. Immunoreactive cytochrome c bands were detected using specific anti-cytochrome c antibody, and visualized using a chemiluminescent second antibody (ECL, Amersham Biosciences, Uppsala, Sweden) in colon tissue from healthy rats (lanes 1 and 2, TNBS−), and from rats 24 hours after induction of colitis with hapten (TNBS, 100 mg/ml) intra-anally (lanes 3 and 4, TNBS+). Note the alteration in electrophoretic mobility of the cytochrome c of colon tissue following induction of colon inflammation (lane 3, Se suppl−; TNBS+) in controls, and the profound protection from changes in cytchrome c provided by high dose selenium dietary supplementation afforded to induced rats (lane 4, Se suppl+; TNBS+, compared to lane 3).

FIGS. 8A and 8B are graphs illustrating the in vitro inhibition of ROS production and stimulation of antioxidant enzyme activity in macrophages by selenium. Murine J774.2 macrophages grown in RPMI medium were exposed to increasing (0.5-30 μM) concentrations of sodium selenite. FIG. 8A shows a concentration-dependent reduction of dihydrodichlorofluorescein (H₂DCF-DA)-sensitive ROS (H₂O₂) production, expressed as DCF units per mg protein, by intracellular fluorescence in a flow cytometer, or total fluorescence using a microfluorometer plate reader. Data were collected from at least 10,000 cells. FIG. 8B shows the stimulation of glutathione peroxidase activity, expressed as the rate of NADPH oxidation per mg protein, in 100 μl cell extract, by low concentrations of sodium selenite. Note that maximum stimulation of GPx activity is achieved at 0.5 μM, while inhibition of ROS production continues throughout the range tested.

FIGS. 9A and 9B are RTPCR and Western blot analysis of the down-regulation of MnSOD transcription and expression by selenium. Murine J774.2 macrophages grown in RPMI medium were exposed to increasing (0.5-30 μM concentrations of sodium selenite for 24 or 72 hours, and analyzed by RTPCR (FIG. 9A) or Western blot (FIG. 9B) for MnSOD expression. FIG. 9A shows the dramatic decrease in MnSOD transcripts from cells exposed to 5 and 10 μM sodium selenite for 24 (upper panels, and open columns of graph) and 72 (lower panels and filled columns of graph) hours incubation. Graph is densitometric expression of MnSOD/GAPDH (housekeeping control gene) transcript ratio. FIG. 9B is a Western blot of immunodetection of MnSOD protein in extracts of cells exposed to sodium selenite for 24 (24 h) or 72 (72 h) hours. Note the absence of detectable MnSOD protein in the cells after 72 hours at 10 μM sodium selenite.

FIGS. 10A and 10B are RTPCR and Western blot analysis of the down-regulation of uncoupling protein 2 (UCP2) transcription and expression by selenium. Murine J774.2 macrophages grown in RPMI medium were exposed to increasing (0.5-30 μM) concentrations of sodium selenite for 24 or 72 hours, and analyzed by RTPCR (FIG. 10A) or Western blot (FIG. 10B) for UCP2 expression. FIG. 10A shows a significant decrease in UCP2 transcripts from cells exposed to as little as 5 μM sodium selenite for 24 (upper panels, and open columns of graph) and 72 (lower panels and filled columns of graph) hours incubation. Graph is densitometric expression of UCP2/GAPDH (housekeeping control gene) transcript ratio. FIG. 10B is a Western blot of immunodetection of UCP2 protein in extracts of cells exposed to sodium selenite for 24 (24 h) or 72 (72 h) hours. Note the greater than 50% reduction in detectable UCP2 protein in the cells after 24 hours at 30 μM sodium selenite.

FIG. 11 is a RTPCR analysis illustrating the down-regulation of MnSOD and UCP2 transcription and expression by selenium in activated macrophages. Murine J774.2 macrophages grown in RPMI medium were activated by exposure to endotoxin (LPS, 10 ng/ml) and INFγ (50 units/ml) (LPS+INFγ+), exposed to increasing (0.5-5 μM) concentrations of sodium selenite for 24 and analyzed by RTPCR for MnSOD and UCP2 expression. Activation clearly upregulates MnSOD expression (lane LPS+INFγ+, Se−), without effecting UCP2 expression (lane LPS+INFγ+, Se−). Exposure to as little as 1 μM sodium selenite for 24 hours (lane LPS+INFγ+, Se 1 μM) was effective in reducing both UCP2 and MnSOD expression levels. Graph is densitometric expression of MnSOD and UCP2/GAPDH (housekeeping control gene) transcript ratio.

FIGS. 12A and 12B illustrate the downregulation of transcription factor NF-κB binding activity by selenium in activated macrophages. Murine J774.2 macrophages grown in RPMI medium with or without increasing (0.5-5 μM) concentrations of sodium selenite for 72 hours were activated by 6 hours exposure to endotoxin (LPS, 10 ng/ml) and INF? (50 units/ml) (LPS+INFγ+), lysed, and nuclear extracts prepared. NF-κB translocation to the nucleus was assessed by binding activity of the nuclear extracts as measured by the electromobility shift assay (EMSA) using a specific ³²P labeled NF-κB consensus oligonucleotide. FIG. 12A-Autoradiographs of reacted nuclear extracts separated on non-denaturing acrylamide gel show a dramatic effect of as little as 0.5 μM selenium on NF-κB translocation in nuclei of activated macrophages (LPS+INFγ+, Se 0.5), while no effect is noticed in nuclei of resting, unactivated macrophages (LPS+INFγ−, Se 0.5-5). Addition of unlabeled oligonucleotide inhibited binding of labeled consensus oligonucleotide (competition). FIG. 12B illustrates the inhibitory effect of 5 μM sodium selenite added to an EMSA assay of NF-κB binding in-vitro 5 minutes before electrophoretic separation.

FIGS. 13A-13C illustrate the attenuation of transcription factor Sp1 binding activity by selenium in activated macrophages. Murine J774.2 macrophages grown in RPMI medium with or without increasing (0.5-5 μM) concentrations of sodium selenite for 72 hours were activated by 6 hours exposure to endotoxin (LPS, 10 ng/ml) and INFγ (50 units/ml) (LPS+INFγ+), lysed, and nuclear extracts prepared. Sp1 translocation to the nucleus was assessed by binding activity of the nuclear extracts, measured by the electromobility shift assay (EMSA) using a specific ³²P labeled Sp-1 consensus oligonucleotide (FIGS. 13A and 13C), and by immunodetection on Western blots (FIG. 13B). FIG. 13A-Autoradiographs of reacted nuclear extracts separated on non-denaturing acrylamide gel show a dramatic effect of as little as 5 μM selenium on Sp-1 binding in nuclei of activated macrophages (LPS+INFγ+, Se 5, lower panel), and of resting, unactivated macrophages (LPS+INFγ−, Se 5+, upper panel). Addition of unlabeled oligonucleotide inhibited binding of labeled consensus oligonucleotide (competition). FIG. 13B shows the immunodetection of SP-1 on a Western blot, demonstrating the lack of effect of sodium selenite on Sp-1 gene expression. FIG. 13C illustrates the inhibitory effect of 5 μM sodium selenite added to an EMSA assay of SP-1 binding in-vitro 5 minutes before electrophoretic separation.

FIGS. 14A and 14B are graphs illustrating the effect of selenium on survival of activated macrophages. Murine J774.2 macrophages grown in RPMI medium were activated by 24 hours exposure to endotoxin (LPS, 10 ng/ml) and INFγ (50 units/ml) (LPS+INFγ+), and exposed to 10 μM sodium selenite (Se+) during (FIG. 14A) activation or after (FIG. 14B) activation. Viability was expressed as cell-membrane integrity, measured by flow cytometry using the non-permeant dye PI (cell viability). Note the sodium selenate-mediated decrease (up to 60%) in cell viability in both activated (LPS+INFγ+) and inactivated (LPS+INFγ−) cells (14A, and 14B, columns d and c, compared with columns a and b).

FIGS. 15A and 15B illustrate the character of selenium mediated cell death in macrophages. Murine J774.2 macrophages grown in RPMI medium were exposed to 10 μM sodium selenite (Se+) for 24 hours, and character of cell death was assessed by dual staining with annexin V-FITC and PI by flow cytometry. Note the increased cell death with sodium selenate (FIG. 15A, control vs 10 μM Se), and the incorporation of both annexin V-FITC and PI in the majority of dead cells (FIG. 14A, black column), indicating necrotic rather than apoptotic death Dot plot of cells showing low PI uptake (FIG. 15B, quadrant c control vs quadrant c selenium) did not have increased annexin V-FITC uptake, negating apoptotic death in these cells.

FIGS. 16A and 16B show the inhibition of ROS production and mitochondrial membrane potential (MMP) by selenium in activated macrophages. Murine J774.2 macrophages grown in RPMI medium were activated by 24 hours exposure to endotoxin (LPS, 10 ng/ml) and INF? (50 units/ml) (LPS+INF?+), followed by exposure to 10 μM sodium selenite (Se+). ROS production, expressed as DCF units per mg protein (FIG. 16A), and MMP, expressed as incorporation of the potential-sensitive probe CMTMRos (FIG. 16B) were evaluated by fluorescent flow cytometry. Note that both ROS production (FIG. 16A) and MMP (FIG. 16B) were strongly inhibited by sodium selenate in activated (columns a and c) and resting (columns b and d) macrophages.

FIG. 17 shows the upregulation of mitochondrial transcription factors by high dose dietary selenium supplementation. The upper panel is a gel of the products of RT-PCR analysis of mitochondrial Transcription Factor A (mtTFA) and Nuclear Respiratory Factor (NRF), as well as housekeeping genes 18S RNA and GADPH expression in pooled RNA from rats receiving three weeks of high dose selenium dietary supplementation of 16 ppm sodium selenite in the drinking water (lanes 2 and 4, Se suppl+) or no selenium dietary supplementation (lanes 1 and 3, Se suppl−). The abundance of mitochondrial transcription factor transcripts in colon tissue from selenium supplemented healthy rats (lane 1 and 2, TNBS−), and from rats 24 hours after induction of colitis with hapten (TNBS, 100 mg/ml) intra-anally (lanes 3 and 4, TNBS+) was detected using specific primers, and gel-separated PCR products visualized by EtBr staining. Note the strong increase in mtTFA and NRF1 expression (see densitometry values in the lower panel) following selenium supplementation, in both the healthy (lane 2) and colitis-induced rats (lane 4).

FIG. 18 is a schematic depiction of a proposed molecular mechanism of selenium's attenuation of inflammatory response. Dotted arrows indicate the anti-inflamatory effects of selenium acting at the levels of NRF1 and mtTFA transcription, Sp-1 and NF-κB binding, MnSOD and UCP2 expression, leading to reduced ROS H₂O₂ production, and decreased macrophage viability.

### DESCRIPTION OF THE PREFERRED EMBODIMENTS

The present invention is of a method for treatment of inflammatory disease, particularly IBD, by short-term, high dose selenium supplementation, which can be used to efficiently treat a variety of inflammatory disorders. Specifically, the method and article of manufacture of the present invention can be used to raise tissue selenium levels in a subject, in order to alleviate or prevent inflammation-associated cellular damage and inflammatory disease.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Inflammatory diseases of the gastrointestinal tract are typically treated with anti-inflammatory or immunosuppressive agents, and/or surgery (colectomies). However, as described in the Background section hereinabove, the drugs presently available provide, at best, only partial relief and are associated with severe side effects such as nausea, dizziness, anemia, leukopenia and skin rashes. On the other hand, surgery is a severe, poorly tolerated, and often only temporarily effective solution.

Nutritional therapy and dietary supplementation have been recommended for inflammatory disease. Since low levels of nutritional selenium intake have been correlated with a number of inflammatory and cancerous conditions, selenium supplementation has been included in a number of therapeutic nutritional compositions. However, as described in detail in the Background section hereinabove, selenium is included in such compositions as one of a number of anti-oxidant agents, and is provided in low doses only, in the range of 50-1000 μg/day, typically not exceeding 200 μg/day (see U.S. Pat. No. 6,524,619 to Pearson et al, U.S. Pat. No. 5,156,852 to La Haye et al., U.S. Pat. No. 6,444,221 to Shapiro, U.S. Pat. No. 5,405,613 to Rowland, U.S. Pat. No. 5,639,482 to Crary, U.S. Pat. Nos. 6,242,010 and 6,303,651 to Hersch, U.S. Pat. No. 6,214,373 to Snowden, U.S. Pat. Nos. 6,420,342 and 6,548,483 to Hageman et al, U.S. Pat. No. 5,444,044 to Garleb et al, and U.S. Pat. Nos. 5,925,314, 6,468,987 and 5,780,451 to DeMichele et al, all incorporated herein by reference).

None of the abovementioned prior art documents actually disclose the use of selenium for treatment of inflammatory bowel disease. Most of the prior art merely describes the formulation of various selenium-containing compositions and supplements, without providing examples of their use or efficacy in treating inflammation, in laboratory or clinical conditions (see, for example, U.S. Pat. Nos. 5,405,852; 5,405,613; 5,661,123; 5,922,766, 5,939,394 and 6,495,170). For example, U.S. Pat. No. 6,444,221 to Shapiro discloses one anecdotal case of a mild improvement in inflammation using a PABA/methionine/Vitamin E formulation lacking selenium. U.S. Pat. No. 5,444,044 to Garleb, et al., and U.S. Pat. Nos. 5,953,314, 6,468,987, and 5,780,451 to Demichele et al, which teach a moderate effect of fish oil and antioxidants on inflammatory parameters, also fail to disclose any use of selenium in the compositions used. Selenium, in combination with vitamin E (see U.S. Pat. Nos. 5,639,482 and 5,156,852) has been proposed for treatment of macular degeneration and diabetic retinopathy. Only anecdotal evidence of effectiveness was provided (U.S. Pat. No. 5,639,482 to Crary), in which transient improvement in the symptoms of diabetic retinopathy was achieved. Thus, none of the abovementioned prior art teaches administration of high doses of selenium for inflammatory disease.

Dietary selenium supplementation has been investigated in studies of cancer, aging, and oxidative stress. For example, Yan et al (Anticancer Res 1999;19:1337-42) demonstrated that 2.5 ppm sodium selenite in water inhibited tumor size and incidence of metastases from melanoma cells in mice. Thirunavukkarasu and his co-workers have reported effective reduction of DEN and Phenobarbital induced hepatocarcinoma with 1-4 ppm selenite in the drinking water of rats (Thirunavukkarasu et al, Biomed Pharmacother 2003; 57:416-21; Biomed Pharmacother 2003; 57:417-23; Pharmacol Res 2003;48:167-73; Cell Biochem Func 2003;21:147-53; J Cell Biochem 2003;88:578; Cell Biochem Func 2002;20:374-56; and 2001; 19;365-71). Sivaram et al (Comp Bio Physio C Toxic Pharmacol 2003;134:397-401) reported reduction of alcohol-induced oxidative stress and lipid peroxidation in livers of guinea pigs receiving 0.5 mg/kg body weight per day sodium selenite for 45 days duration. Wong et al. (Anticancer Res 2001;21:393-402) demonstrated reduction of malignant lymphoproliferative pathologies in OF1 mice drinking water supplemented with 2.5 ppm sodium selenite. However, studies with myocarditic Coxsackie virus B3 virus-induced inflammation in mice showed no effect of selenium supplementation (5 ppm in the water) on inflammatory lesions and necrosis (Ilback et al Bio Trace Elem Res 1998;63:51-66). Similarly conflicting results were reported by Reddy et al (Int J Mol Med, 2000;5:327-30), who found that selenomethionine at up to 5.4 ppm in water, was ineffective as an anti-cancer treatment in AOM-induced colon cancer.

While reducing the present invention to practice, it was surprisingly uncovered that short-term administration of high dose dietary selenium, in the form of sodium selenite, effectively prevented tissue damage, myeloperoxidase activity, and mitochondrial damage associated with hapten-induced colon inflammation in rats. As described in the Examples section hereinbelow, selenium supplementation was provided by high dose sodium selenite-supplementation in the drinking water, ad libitum for 20 days. This is the first demonstration of anti-inflammatory effects of short term, high dose dietary selenium supplementation.

Thus, according to the present invention there is provided a method for treating a subject having an inflammatory disease or condition, the method comprising administering to the subject selenium in a daily amount of about 0.1 μmol to about 100 μmol per kg body weight, for at least one day, thereby treating the subject for the inflammatory disease or condition.

According to the present invention, selenium can be administered as a constituent of a selenium source. The source of selenium can be from organic or inorganic selenium. Suitable sources of selenium from inorganic selenium include, but are not limited to, sodium selenite, sodium selenide, sodium selenate, selenic acid, selenium sulfide, selenium oxychloride, selenium dioxide, hydrogen selenide and selenious acid. Suitable sources of organic selenium include, but are not limited to dimethyl selenium, trimethyl selenium, selenomathionine, selenocysteine, seleno-methyl selenocysteine, selenocystathionine, selenotaurine, selenodiglutathione, allyl selenocysteine, propyl selenocysteine, selenoethionine and selenocystamine. Such selenium-containing compounds are commercially available from, for example, Omkar Chemicals, Maharashta, INDIA for organoselenium compounds, and Sigma-Aldrich Corp., St Louis, Mo., USA for elemental selenium and inorganic selenium compounds.

While reducing the present invention to practice, it was demonstrated that short-term, high dose dietary selenium supplementation is effective in treating and preventing inflammatory disease. Selenium supplementation was provided therapeutically, more than an order of magnitude in excess of the RDA. Thus, according to the present invention, the method of treating a subject for inflammatory disease or condition is effected by administering about 0.1 μmol to about 100 μmol selenium per kg body weight, for at least one day. Dosage unit equivalents expressed in mass of selenium per kg body weight are easily calculated: Elemental selenium has an atomic mass of 78.9 atomic mass units, thus, 1.0 μmol selenium, alone or in a compound (such as sodium selenite, Na₂SeO₃) has a mass of 78.9 micrograms. Therefore, the range of about 0.1 μmol to about 100 μmol selenium per kg body weight is equivalent to about 7.89 micrograms to about 7.89 milligrams selenium per kg body weight. In one embodiment, the method is effected by administering about 2 μmol to about 50 μmol selenium per kg body weight. In another, preferred embodiment, about 10 μmol to about 20 μmol per kg body weight are administered.

In the context of the present invention, it is important to note that there is substantial lack of clarity regarding the toxicity and carcinogenicity of selenium. Long-term exposure to exceedingly high levels of selenium has been found to lead to toxicity in some cases. It had been observed that the inhabitants of geographical areas having high selenium levels in the soil and food (average lifetime daily intake of greater than 1000 microgram selenium) developed biochemical (alterations in liver function) and clinical (hair and nail loss, morphological changes in nails) symptoms (Yang, et al J Trace Elem Electrolyte Health Dis 1989;3, 77-87; and 123-30). However, due to the low sample numbers in these epidemiological studies, and the lack of experimental support, the significance of the purported biochemical changes has been questioned. Recent studies with mice demonstrated that toxicity was observed at 60 and 90 days only with dietary supplementation equivalent to greater than 10 mg selenium per kg body weight per day (as selenocysteine) (Hasegama T, Arch Toxicol 1994;68:91-95). Regarding the carcinogenicity of selenium, studies sponsored by the National Toxicology Institute have indicated that exceedingly high concentrations of selenium (greater than 20 mg per kg body weight per day) for long periods of time (greater than 100 weeks) were required to produce significant carcinogenesis in mice and rats (National Toxicology Program Tech Report, Ser 1980;194:1-197). It is significant to note that some of the animals studied (male mice) were unaffected at even higher doses. National Toxicology Program of the US has reported (TOX-38, 1994) that long term supplementation of mice and rats with up to 0.8 mg/kg body weight selenium as selenate or selenite produced no observable adverse effects. Recommendations for changes in the RDA and allowable levels vary considerably with different assessments (see, for example, Koller, L D et al, Can J Vet Res 1986;50:297-306).

Thus, the short-term, high dose dietary selenium supplementation of the present invention avoids toxic or carcinogenic levels of exposure observed with long term exposure. According to the present invention, the administration of selenium is for at least one day. In a preferred embodiment, the administration is carried out twice daily. In another preferred embodiment, the administration is for not more than 21 days. In a more preferred embodiment, the administration is for not more than 12 months. Methods and conditions of administration, methods for determining treatment regimens, and methods for monitoring the effectiveness of treatment are described in detail hereinbelow.

According to the method of the present invention, treatment of a subject having an inflammatory disease or condition is effected by short-term administration of high dose dietary selenium supplementation.

Examples of inflammatory diseases include, but are not limited to, hypersensitivity, autoimmune disease, infectious disease, graft rejection, allergic disease, inflammatory musculo-skeletal disease, gut-related inflammatory disease, neurological inflammatory disease, inflammatory cardiovascular disease, injury, idiopathic inflammatory disease and inflammation of unknown etiology.

According to one preferred embodiment of the present invention short-term administration of high dose dietary selenium supplementation is employed to treat gut-related inflammatory disease, such as colitis, as described in Example 2 of the Examples section, below. According to another preferred embodiment, the gut-related inflammatory disease is an inflammatory bowel disease, mucositis, necrotizing enterocolitis, aphthous stomatitis, pharyngitis, esophagitis, peptic ulcers, gingivitis and periodontotitis. According to a more preferred embodiment, the inflammatory bowel disease is Crohn's disease, ulcerative colitis, indeterminate colitis and infectious colitis.

According to yet a further embodiment of the present invention, short-term administration of high dose dietary selenium supplementation is used to treat an inflammation associated with hypersensitivity.

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and delayed type hypersensitivity.

Examples of Type 1, or immediate hypersensitivity include, but are not limited to, asthma.

Examples of Type II hypersensitivity include, but are not limited to, rheumatoid diseases, rheumatoid autoimmune diseases, rheumatoid arthritis (Krenn V. et al., Histol Histopathol 2000 July;15:791), spondylitis, ankylosing spondylitis (Jan Voswinkel et al., Arthritis Res 2001; 3: 189), systemic diseases, systemic autoimmune diseases, systemic lupus erythematosus (Erikson J. et al., Immunol Res 1998;17:49), sclerosis, systemic sclerosis (Renaudineau Y. et al., Clin Diagn Lab Immunol. 1999 March;6:156); Chan O T. et al., Immunol Rev 1999 June;169:107), glandular diseases, glandular autoimmune diseases, pancreatic autoimmune diseases, diabetes, Type I diabetes (Zimmet P. Diabetes Res Clin Pract 1996 October;34 Suppl:S125), thyroid diseases, autoimmune thyroid diseases, Graves' disease (Orgiazzi J. Endocrinol Metab Clin North Am 2000 June;29:339), thyroiditis, spontaneous autoimmune thyroiditis (Braley-Mullen H. and Yu S, J Immunol 2000 December 15;165:7262), Hashimoto's thyroiditis (Toyoda N. et al., Nippon Rinsho 1999 August;57:1810), myxedema, idiopathic myxedema (Mitsuma T. Nippon Rinsho. 1999 August;57:1759); autoimmune reproductive diseases, ovarian diseases, ovarian autoimmunity (Garza K M. et al., J Reprod Immunol 1998 February;37:87), autoimmune anti-sperm infertility (Diekman A B. et al., Am J Reprod Immunol. 2000 March;43:134), repeated fetal loss (Tincani A. et al., Lupus 1998;7 Suppl 2:S107-9), neurodegenerative diseases, neurological diseases, neurological autoimmune diseases, multiple sclerosis (Cross A H. et al., J Neuroimmunol 2001 January 1;1 12:1), Alzheimer's disease (Oron L. et al., J Neural Transm Suppl. 1997;49:77), myasthenia gravis (Infante A J. And Kraig E, Int Rev Immunol 1999;18:83), motor neuropathies (Kornberg A J. J Clin Neurosci. 2000 May;7:191), Guillain-Barre syndrome, neuropathies and autoimmune neuropathies (Kusunoki S. Am J Med Sci. 2000 April;319:234), myasthenic diseases, Lambert-Eaton myasthenic syndrome (Takamori M. Am J Med Sci. 2000 April;319:204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January;156:23); neuropathies, dysimmune neuropathies (Nobile-Orazio E. et al., Electroencephalogr Clin Neurophysiol Suppl 1999;50:419); neuromyotonia, acquired neuromyotonia, arthrogryposis multiplex congenita (Vincent A. et al., Ann N Y Acad Sci. 1998 May 13;841:482), cardiovascular diseases, cardiovascular autoimmune diseases, atherosclerosis (Matsuura E. et al., Lupus. 1998;7 Suppl 2:S135), myocardial infarction (Vaarala O. Lupus. 1998;7 Suppl 2:S132), thrombosis (Tincani A. et al., Lupus 1998;7 Suppl 2:S107-9), granulomatosis, Wegener's granulomatosis, arteritis, Takayasu's arteritis and Kawasaki syndrome (Praprotnik S. et al., Wien Klin Wochenschr 2000 August 25;1 12 (15-16):660); anti-factor VIII autoimmune disease (Lacroix-Desmazes S. et al., Semin Thromb Hemost.2000;26 (2): 157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Intern (Paris). 2000 May;151 (3):178); antiphospholipid syndrome (liamholz R. et al., J Clin Apheresis 1999;14 (4):171); heart failure, agonist-like beta-adrenoceptor antibodies in heart failure (Wallukat G. et al., Am J Cardiol. 1999 June 17;83 (12A):75H), thrombocytopenic purpura (Moccia F. Ann Ital Med Int. 1999 April-June;14 (2):114); hemolytic anemia, autoimmune hemolytic anemia (Efremov D G. et al., Leuk Lymphoma 1998 January;28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January;23 (1):16), celiac disease (Landau Y E. and Shoenfeld Y. Harefuah 2000 January 16;138 (2):122), autoimmune diseases of the musculature, myositis, autoimmune myositis, Sjogren's syndrome (Feist E. et al., Int Arch Allergy Immunol 2000 September;123 (1):92); smooth muscle autoimmune disease (Zauli D. et al., Biomed Pharmacother 1999 June;53 (5-6):234), hepatic diseases, hepatic autoimmune diseases, autoimmune hepatitis (Manns M P. J Hepatol 2000 August;33 (2):326) and primary biliary cirrhosis (Strassburg C P. et al., Eur J Gastroenterol Hepatol. 1999 June;11 (6):595).

According to a yet another embodiment, the method of the present invention is employed to treat Type IV or T lymphocyte mediated hypersensitivity.

Examples of Type IV or T cell mediated hypersensitivity, include, but are not limited to, rheumatoid diseases, rheumatoid arthritis (Tisch R, McDevitt H O. Proc Natl Acad Sci USA 1994 January 18;91 (2):437), systemic diseases, systemic autoimmune diseases, systemic lupus erythematosus (Datta S K., Lupus 1998;7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March;92 (1):77); ovarian diseases (Garza K M. et al., J Reprod Immunol 1998 February;37 (2):87), prostatitis, autoimmune prostatitis (Alexander R B. et al., Urology 1997 December;50 (6):893), polyglandular syndrome, autoimmune polyglandular syndrome, Type I autoimmune polyglandular syndrome (Hara T. et al., Blood. 1991 March 1;77 (5):1127), neurological diseases, autoimmune neurological diseases, multiple sclerosis, neuritis, optic neuritis (Soderstrom M. et al., J Neurol Neurosurg Psychiatry 1994 May;57 (5):544), myasthenia gravis (Oshima M. et al., Eur J Immunol 1990 December;20 (12):2563), stiff-man syndrome (Hiemstra H S. et al., Proc Natl Acad Sci USA 2001 March 27;98 (7):3988), cardiovascular diseases, cardiac autoimmunity in Chagas' disease (Cunha-Neto E. et al., J Clin Invest 1996 October 15;98 (8):1709), autoimmune thrombocytopenic purpura (Semple J W. et al., Blood 1996 May 15;87 (10):4245), anti-helper T lymphocyte autoimmunity (Caporossi A P. et al., Viral Immunol 1998;11 (1):9), hemolytic anemia (Sallah S. et al., Ann Hematol 1997 March;74 (3):139), hepatic diseases, hepatic autoimmune diseases, hepatitis, chronic active hepatitis (Franco A. et al., Clin Immunol Immunopathol 1990 March;54 (3):382), biliary cirrhosis, primary biliary cirrhosis (Jones D E. Clin Sci (Colch) 1996 November;91 (5):551), nephric diseases, nephric autoimmune diseases, nephritis, interstitial nephritis (Kelly C J. J Am Soc Nephrol 1990 August;1 (2):140), connective tissue diseases, ear diseases, autoimmune connective tissue diseases, autoimmune ear disease (Yoo T J. et al., Cell Immunol 1994 August;157 (1):249), disease of the inner ear (Gloddek B. et al., Ann N Y Acad Sci 1997 December 29;830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocyte mediated imunity and cytotoxic T lymphocyte-mediated immunity.

According to a preferred embodiment of the present invention, short-term administration of high dose dietary selenium supplementation is employed to treat an inflammation associated with an inflammatory cardiovascular disease.

Examples of inflammatory cardiovascular disease include, but are not limited to atherosclerosis (Matsuura E. et al., Lupus. 1998;7 Suppl 2:S135), myocardial infarction (Vaarala O. Lupus. 1998;7 Suppl 2:S132), thrombosis (Tincani A. et al., Lupus 1998;7 Suppl 2:S107-9), Wegener's granulomatosis, Takayasu's arteritis, Kawasaki syndrome (Praprotnik S. et al., Wien Klin Wochenschr 2000 August 25;112 (15-16):660), anti-factor VIII autoimmune disease (Lacroix-Desmazes S. et al., Semin Thromb Hemost.2000;26 (2):157), necrotizing small vessel vasculitis, microscopic polyangiitis, Churg and Strauss syndrome, pauci-immune focal necrotizing and crescentic glomerulonephritis (Noel L H. Ann Med Intern (Paris). 2000 May;151 (3):178), antiphospholipid syndrome (Flamholz R. et al., J Clin Apheresis 1999;14 (4):171), antibody-induced heart failure (Wallukat G. et al., Am J Cardiol. 1999 June 17;83 (12A):75H), thrombocytopenic purpura (Moccia F. Ann Ital Med Int. 1999 April-June;14 (2):114; Semple J W. et al., Blood 1996 May 15;87 (10):4245), autoimmune hemolytic anemia (Efremov D G. et al., Leuk Lymphoma 1998 January;28 (3-4):285; Sallah S. et al., Ann Hematol 1997 March;74 (3):139), cardiac autoimmunity in Chagas' disease (Cunha-Neto E. et al., J Clin Invest 1996 October 15;98 (8):1709) and anti-helper T lymphocyte autoimmunity (Caporossi A P. et al., Viral Immunol 1998;11 (1):9) occlusive disease, atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity in Chagas' disease and anti-helper T lymphocyte autoimmunity.

According to another preferred embodiment of the present invention, short-term administration of high dose dietary selenium supplementation is employed to treat a neurological inflammatory disease.

Examples of inflammatory neurological diseases include, but are not limited to, neurodegenerative disease, multiple sclerosis (Cross A H. et al., J Neuroimmunol 2001 January 1;112 (1-2):1), Alzheimer's disease (Oron L. et al., J Neural Transm Suppl. 1997;49:77), myasthenia gravis (Infante A J. And Kraig E, Int Rev Immunol 1999;18 (1-2):83; Oshima M. et al., Eur J Immunol 1990 December;20 (12):2563), neuropathies, motor neuropathies (Komberg A J. J Clin Neurosci. 2000 May;7 (3):191); Guillain-Barre syndrome and autoimmune neuropathies (Kusunoki S. Am J Med Sci. 2000 April;319 (4):234), myasthenia, Lambert-Eaton myasthenic syndrome (Takamori M. Am J Med Sci. 2000 April;319 (4):204); paraneoplastic neurological diseases, cerebellar atrophy, paraneoplastic cerebellar atrophy and stiff-man syndrome (Hiemstra H S. et al., Proc Natl Acad Sci units S A 2001 March 27;98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January;156 (1):23); dysimmune neuropathies (Nobile-Orazio E. et al., Electroencephalogr Clin Neurophysiol Suppl 1999;50:419); acquired neuromyotonia, arthrogryposis multiplex congenita (Vincent A. et al., Ann N Y Acad Sci. 1998 May 13;841:482), neuritis, optic neuritis (Soderstrom M. et al., J Neurol Neurosurg Psychiatry 1994 May;57 (5):544) and stiff man syndrome.

According to yet another preferred embodiment of the present invention, short-term administration of high dose dietary selenium supplementation is employed to treat an inflammation associated with an infectious disease.

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion disease.

According to another preferred embodiment of the present invention, short-term administration of high dose dietary selenium supplementation is employed to treat an inflammation associated with graft rejection.

Examples of diseases or conditions associated with graft rejection include, but are not limited to, chronic graft rejection, subacute graft rejection, hyperacute graft rejection, acute graft rejection and graft versus host disease.

Types of grafts whose rejection can be treated by the method of the present invention include, but are not limited to, syngeneic grafts, allografts and xenografts.

According to a preferred embodiment of the present invention, short-term administration of high dose dietary selenium supplementation is employed to treat an inflammation associated with an allergic disease.

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

According to a preferred embodiment of the present invention, short-term administration of high dose dietary selenium supplementation is employed to treat an inflammation associated with an inflammatory musculoskeletal disease.

Examples of inflammatory musculoskeletal disease include, but are not limited to autoimmune myositis, primary Sjogren's syndrome, smooth muscle autoimmune disease, rheumatoid arthritis, ankylosing spondylitis, muscle inflammation, myositis, tendon inflammation, tendinitis, ligament inflammation, cartilage inflammation, joint inflammation, synovial inflammation, carpal tunnel syndrome and bone inflammation.

According to another preferred embodiment of the present invention, short-term administration of high dose dietary selenium supplementation is employed to treat an inflammation associated with an autoimmune disease.

Examples of autoimmune diseases include, but are not limited to, Type 1 diabetes (Castano L. and Eisenbarth G S. Ann. Rev. Immunol. 8:647; Zimmet P. Diabetes Res Clin Pract 1996 October;34 Suppl:S125), autoimmune thyroid diseases, Graves' disease (Orgiazzi J. Endocrinol Metab Clin North Am 2000 June;29 (2):339; Sakata S. et al., Mol Cell Endocrinol 1993 March;92 (1):77), spontaneous autoimmune thyroiditis (Braley-Mullen H. and Yu S, J Immunol 2000 December 15;165 (12):7262), Hashimoto's thyroiditis (Toyoda N. et al., Nippon Rinsho 1999 August;57 (8):1810), idiopathic myxedema (Mitsuma T. Nippon Rinsho. 1999 August;57 (8): 1759), ovarian autoimmunity (Garza K M. et al., J Reprod Immunol 1998 February;37 (2):87), autoimmune anti-sperm infertility (Diekman A B. et al., Am J Reprod Immunol. 2000 March;43 (3):134), autoimmune prostatitis (Alexander R B. et al., Urology 1997 December;50 (6):893), Type I autoimmune polyglandular syndrome (Hara T. et al., Blood. 1991 March 1;77 (5):1127), autoimmune gastrointestinal diseases such as celiac disease (Landau Y E. and Shoenfeld Y. Harefuah 2000 January 16;138 (2):122) and ileitis, autoimmune cutaneous diseases such as pemphigus vulgaris, bullous pemphigoid and pemphigus foliaceus, autoimmune hepatic diseases such as hepatitis, autoimmune chronic active hepatitis (Franco A. et al., Clin Immunol Immunopathol 1990 March;54 (3):382), primary biliary cirrhosis (Jones D E. Clin Sci (Colch) 1996 November;91 (5):551; Strassburg C P. et al., Eur J Gastroenterol Hepatol. 1999 June;11 (6):595) autoimmune hepatitis (Manns M P. J Hepatol 2000 August;33 (2):326), primary Sjogren's syndrome (Feist E. et al., Int Arch Allergy Immunol 2000 September;123 (1):92) and smooth muscle autoimmune disease (Zauli D. et al., Biomed Pharmacother 1999 June;53 (5-6):234), nephritis, autoimmune interstitial nephritis (Kelly C J. J Am Soc Nephrol 1990 August;1 (2):140), autoimmune connective tissue diseases such as ear disepases, autoimmune ear diseases (Yoo T J. et al., Cell Immunol 1994 August;157 (1):249) and autoimmune diseases of the inner ear (Gloddek B. et al., Ann N Y Acad Sci 1997 December 29;830:266), and autoimmune systemic diseases such as systemic lupus erythematosus (Erikson J. et al., Immunol Res 1998;17 (1-2):49) and systemic sclerosis (Renaudineau Y. et al., Clin Diagn Lab Immunol. 1999 March;6 (2):156); Chan O T. et al., Immunol Rev 1999 June;169:107).

While reducing the present invention to practice, a direct effect of selenium on reactive species production, mitochondrial membrane integrity, and viability of macrophages was surprisingly uncovered. As described in detail in Example III of the Results section hereinbelow, short term exposure to selenium decreased MnSOD expression, inhibited peroxide production, and caused cell death in resting and activated J774.2 macrophages in vitro. Reduction in the number and activity of macrophages by selenium treatment of the present invention can be significant in treatment and prevention of inflammatory diseases characterized by infiltration of macrophages.

According to a preferred embodiment of the present invention, short-term administration of high dose dietary selenium supplementation is employed to treat a subject having a macrophage mediated inflammatory disease or condition. Some non-Limiting examples of macrophage-mediated disease or conditions are Gulillian-Barre syndrome, histiocytosis, crescentic glomerulonephritis and atherosclerosis.

Further, administration of selenium according to the methods of the present invention can be used to reduce inflammatory activity in immune cells in culture and in vivo. Thus, according to the present invention there is provided a method for downregulating an activity of an inflammatory immune cell, the method comprising exposing the cell to a concentration of selenium sufficient to downregulate an inflammatory process in the cell, thereby downregulating an activity of the inflammatory immune cell. In one preferred embodiment exposure of the immune cells is in vivo.

As used herein, the phrase inflammatory immune cell is defined as a cell of the immune system participating in an inflammatory reaction or process. Non-limiting examples of inflammatory immune cells are T-lymphocytes, dendritic cells, eosinophils, macrophages, granulocytes, monocytes and macrophages. In one embodiment, the inflammatory immune cells are macrophages. In a preferred embodiment, the macrophages are activated inflammatory immune cells. Inflammatory activity of immune cells is defined as production of proinflammatory cytokines, antigen presentation, respiratory bursts and the like.

While reducing the present invention to practice, it was observed, for the first time, that exposure of activated immune cells (macrophages-see Example III) to selenium reduces the expression of mitochondrial proteins involved in reactive oxygen species production, such as MnSOD and UCP2. Thus, in a preferred embodiment, the inflammatory process is reactive oxygen species production and the downregulation is via reduced expression of mitochondrial proteins. In another embodiment, the concentration of selenium is about 0.1 μM to about 1000 μM, more preferred, about 0.5 μM to about 50 μM.

According to the present invention, short-term administration of high dose dietary selenium supplementation is employed to treat a subject having an inflammatory disease or condition. The new method may be carried out by administering selenium, or a selenium source as disclosed above, to the subject. This method may be carried out by administering the selenium or selenium source, in either a solid dosage form, or in a liquid dosage form.

It should be noted that the term “treatment” also includes amelioration or alleviation of a pathological condition and/or one or more symptoms thereof, curing such a condition, or preventing the genesis of such a condition.

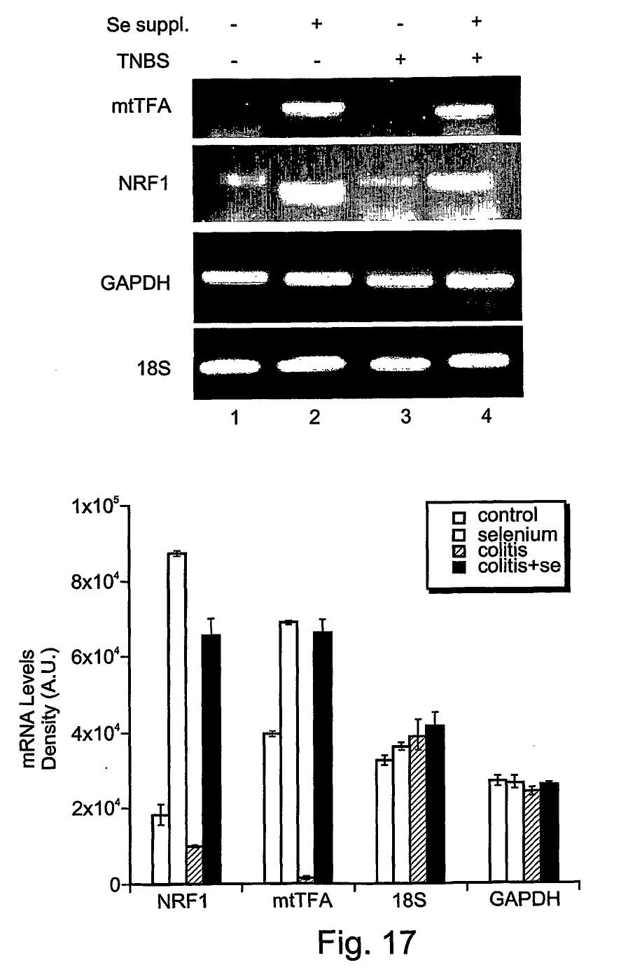

The present invention can be used to treat diseases or conditions associated with mitochondrial dysfunction. While reducing the present invention to practice, it was uncovered, for the first time, that short-term, high dosage dietary selenium supplementation prevents mitochondrial damage in hapten-induced colitis, preventing the impairment of tissue levels of mitochondrial factors NRF1 and mtTFA in the effected colon (Example IV, and FIG. 17). Further, it was uncovered, for the first time, that short term, high dose dietary selenium supplementation elevates the tissue levels of these mitochondrial transcription factors in normal colon tissue from healthy, control rats. Thus, according to one aspect of the present invention, there is provided a method for treating a subject having a disease or condition associated with altered mitochondrial function comprising administering to the subject selenium in a daily amount of about 0.1 μmol to about 100 μmol per kg body weight, for at least one day, thereby treating the subject for the disease or condition.

Altered mitochondrial function, and/or mitochondrial dysfunction, have been implicated in the pathogenesis of a growing number of disease entities. In a recent review of the clinical features of mitochondrial disorders, Simon et al (Ann Rev Med, 1999; 50:111-27) linked mitochondrial dysfunction to a variety of recognizable clinical syndromes, including seizures, strokes, optic atrophy, neuropathy, myopathy, cardiomyopathy, sensorineural hearing loss, diabetes mellitus, and age-related neurodegenerative disease. Many of the mitochondrial-related diseases have been identified through deleterious mutations in mitochondrial proteins (for a recent review, see Schon et al, J Clin Invest 2003, 111:313-12) or factors associated with mitochondrial metabolism and biogenesis (for general reviews see Zagosin et al, Vopr Med Khim 2002; 48:321-36, Ohta, Curr Med Chem 2003;10:2485-94, and Duchen, Diabetes, 2004;54, Supl:S96-102). Steps of pathogenesis in mitochondrial-related disease, include the mutations of nuclear or mitochondrial genes, disturbances of mitochondrial protein synthesis, dissipation of proton membrane potential, opening of a permeability transition pore, releasing of procaspases, cytochrome c, and other proapoptotic molecules, and chromatin fragmentation and apoptotic cell death. Table I lists some of the mitochondrial-related diseases and their genetic origin:

TABLE 1
Neurodegenerative disorders with mitochondrial involvement

| Neurodegenerative diseases due to: | Mutated gene product |
|---|---|
| Primary mutations in mtDNA | |
| Leigh syndrome | CCO III, ND5, tRNA^{Trp}, |
| | tRNA^{Val} |
| LHON/Parkinsonism/Dystonia. | Complex I mcDNA-encoded |
| | subunits |
| Motor neuron disease | CCO I |
| NARP/MILS | ATPase 6 |
| Parkinsonism | 125 rRNA |
| Nuclear gene mutations | |
| in mitochondrion-targeted | |
| proteins affecting OXPHOS | |
| Leigh syndrome with complex | Complex I nDNA-encoded |
| I deficiency | subunits |
| Leigh syndrome with complex | SDH flavoprotein |
| II deficiency | |
| Leigh syndrome with complex | SURF1, SCO2 |
| IV deficiency | |
| Leigh syndrome with PDH | PDH E1-α subunit, |
| deficiency | |
| Nuclear gene mutations in | |
| other mitochondrion-targeted | |
| proteins | |
| ALS | CU, Zn-SOD |
| Friedreich ataxia | Frataxin |
| Hereditary spastic paraplegia | Paraplegin, HSP60 |
| Mohr-Tranebjaerg syndrome | Deafness/dystonia protein-1 |
| | (TIMM8A) |
| Wilson disease | Cu-transporting ATPase |
| | (ATP7B) |
| Nuclear gene mutations in | |
| non-mitochondrion-targeted proteins | |
| AD | ABPP, presenilin-1, |
| | presenilin-2 |
| HD | Huntingrin |
| PD | Parkin, α-synuclein |
| PSP | Tau protein |
| Putative secondary mitochondrial | |
| involvement | |
| Sporadic AD | Unknown |
| Sporadic ALS | Unknown |
| Sporadic PD | Unknown |

LHON, Leber hereditary optic neuropathy; NARP, neuropathy, ataxia, and retinitis pigmentosa; MILS, maternally inherited Leigh syndrome.

(from Schon, et al, J Clin Invest, 2003; 111:303-312).

Targeting of the mitochondrial components of the abovementioned pathologies has been proposed as treatment of mitochondrial-related disease. For example, caspase inhibitors, mitochondrial stabilizers, and Na⁺−H⁺ exchanger inhibitors can reduce apoptotic cell death in acute myocardial ischemia (Garg et al, Curr Opin Cardiol 2003;18:372-7). Using another approach, Fosslein (Ann Clin Lab Sci, 2003;33:271-95) and others have proposed gene therapy for mitochondrial-associated diseases identified with genetic lesions in both mitochondrial and nuclear loci.

U.S. Pat. No. 6,140,067 to Anderson et al. discloses a correlation between mitochondrial dysfunction and diabetes mellitus, and teaches assessment of mitochondrial function for diagnosing diabetes. US Patent Application No. 20020049176, also to Anderson et al, further teaches methods for treating mitochondrial-related disease with agents effecting mitochondrial function, such as NRF-1 and PCG, and for the identification of such agents and the genes encoding them. US Patent Application No. 20030124598 to Speigelman discloses PCG coactivator molecules, antisense sequences and antibodies thereto, and their use in treatment of oxidative stress and other mitochondrial-related disease. However, none of the cited prior art teaches or implies the treatment of disease or conditions associated with altered mitochondrial function by high dose dietary selenium supplementation.

While reducing the present invention to practice, the present inventors have shown that high dose dietary selenium supplementation is effective in increasing the-tissue levels of mitochondrial transcription factors NRF-1 and mtTFA in the inflamed colon tissue, while also suppressing inflammatory macrophage activity. Thus, the high dose dietary selenium supplementation of the present invention can be used to treat or prevent tissue damage and necrotic changes resulting from arrest of mitochondrial respiration, loss of mitochondrial DNA and nuclear encoded mitochondrial proteins in inflammatory and other disease states. Upregulation of mitochondrial transcription factors, as effected by the present invention, can also be a powerful tool in the treatment of diseases associated with altered mitochondrial function, such as insulin resistance and diabetes mellitus (Patti et al, PNAS USA 2003;100:8466-71), and other cellular functions associated with NRF-1 and mtTFA, such as heme biosynthesis (Zong et al., PNAS USA, 2002;99:15983-87).

Determination of altered and/or normal mitochondrial function is well known in the art. Anderson et al (US Patent Application No. 20020049176) disclose a number of indicators of mitochondrial function, such as a mitochondrial electron transport chain enzyme, a Krebs cycle enzyme, a mitochondrial matrix component, a mitochondrial membrane component or an ATP biosynthesis factor, mitochondrial number per cell, mitochondrial mass per cell, an ATP biosynthesis factor, the amount of ATP per mitochondrion, the amount of ATP per unit mitochondrial mass, the amount of ATP per unit protein or the amount of ATP per unit mitochondrial protein, free radical production, a cellular response to elevated intracellular calcium, the activity of a mitochondrial enzyme such as, by way of non-limiting example, citrate synthase, hexokinase II, cytochrome c oxidase, phosphofructokinase, glyceraldehyde phosphate dehydrogenase, glycogen phosphorylase, creatine kinase, NADH dehydrogenase, glycerol 3-phosphate dehydrogenase, triose phosphate dehydrogenase or malate dehydrogenase. In other embodiments, the indicator of mitochondrial function is the relative or absolute amount of mitochondrial DNA per cell in the patient. Methods for the assessment of these indicators are also disclosed. Such indicators can be assessed in cells or tissues from a subject, in order to diagnose a disease or condition associated with mitochondrial dysfunction, and, further, in cells or tissue sampled from a subject under treatment with high dose dietary selenium supplementation, in order to monitor response to the treatment, and in order to determine effective dosage levels. For example, increased expression of NRF1 and mtTFA, measured in the RT-PCR assay as described in the Examples section hereinbelow, would indicate a positive response of a subject receiving high dose dietary selenium supplementation. Further, such assays can be localized to samples of the affected tissues and organs.

In one embodiment of the present invention, the disease or condition associated with altered mitochondrial function is Alzheimer's Disease, Parkinson's Disease, Huntington's disease, progressive supranuclear palsy, diabetes mellitus, hyperproliferative disorders such as cancer, tumors and psoriasis, amyotrophic lateral sclerosis (ALS), Friedreich's ataxia, colon cancer, stroke, exercise intolerance and cardiac myopathy.

Further according to the present invention, there is provided an article of manufacture comprising packaging material and at least one selenium unit dosage, the selenium unit dosage comprising a pharmaceutical composition comprising from about 0.005 mmol to about 10 μmol selenium and a pharmaceutically acceptable carrier in a single unit dosage. The pharmaceutical composition is identified as effective for treatment or prevention of an inflammatory disease or condition by a label or insert included in the packaging material, bearing, for example, clinical indications for use, notification of FDA approval, recommended dosages, frequency and modes of administration, contraindications and the like.

As used herein, the term “selenium unit dosage” is defined as a meted portion comprising the pharmaceutical composition comprising the indicated amount of selenium, suitable for administration as indicated. According to the present invention, each unit dosage comprises a pharmaceutical composition comprising from about 0.005 to about 10 mmol selenium. Dosage in mg selenium per kg body weight per day are easily calculated: as described hereinabove, selenium has an atomic mass of 78.9 amu, thus about 0.005 to about 10 mmol selenium is the equivalent of about 0.395 mg to about 789 mg. Calculations of dosage in mass per kg per day, for manufactured drugs and supplements, can made on the basis of a range of weights of normal populations. Thus, considering that the range of normal human weights is about 50 to 100 kg per person, the unit dosage of the present invention is suited to provide from about 0.00789 mg to about 7.89 mg per kg body weight per day for the lower range of body weight (about 50 kg), and up to 0.00789 mg to about 7.89 mg per kg body weight per day for the upper range (about 100 kg) of body weight. For an average body weight of 75 kg, selenium unit dosage comprising about 0.0075 to about 7.5 mmol selenium administered daily provides 0.00789 mg to about 7.89 mg selenium per kg per day. Dosage for children, and animals can be calculated accordingly.

As noted hereinabove, commercially available selenium supplements providing dosages in the range of the RDA for selenium fall far short of the effective dosage required for the high selenium dietary supplementation of the present invention. In order to provide the high selenium dosage without requiring ingesting cumbersome numbers of the available supplements, a high dosage oral selenium supplement is needed. Thus, according to one aspect of the present invention, the high selenium dietary supplementation is provided in the form of a selenium solid oral dosage form unit comprising as an active ingredient from about 0.005 to about 10 mmol of selenium in a volume of less than 1 cm³. The solid oral dosage may be in the form of tablets, caplets, capsules, granules, beads, particles, pellets and the like, not exceeding a volume of 1 cm³. Standard caplet and tablet dimensions for oral dosage are well known in the art, such as the size 00, 0, 1, 2, 3 . . . capsules available from Capsugel, Inc (Greenwood S.C.). Similarly, solid oral dosage form unit fabrication and manufacture are well known in the art (for detailed description see, for example, “Remington's Pharmaceutical Sciences,” Mack Publishing Co., Easton, Pa., and below). Typically, about 1 gram total of a composition, including pharmaceutical carriers, binders, fillers, excipients and the like can be formed into the capsule, caplet, tablet or other oral dosage forms to be easily administered for treatment of humans.

In one preferred embodiment of the present invention, the selenium dosage unit comprises about 0.05 mmol to about 5 mmol selenium. In a more preferred embodiment, the selenium dosage unit comprises about 0.5 mmol to about 2.5 mmol selenium.

In one embodiment of the present invention, the selenium is a constituent of a selenium source, the selenium being an organic and/or inorganic selenium compound. Selenium compounds suitable for use in the present invention are listed in detail hereinabove.

As used herein a “pharmaceutical composition” refers to a preparation of one or more of the active ingredients described herein, either protein or physiologically acceptable salts or prodrugs thereof, with other chemical components such as traditional drugs, physiologically suitable carriers and excipients. The purpose of a pharmaceutical composition is to facilitate administration of a compound or cell to an organism. Pharmaceutical compositions of the present invention may be manufactured by processes well known in the art, e.g., by means of conventional mixing, dissolving, granulating, dragee-marking, levigating, emulsifying, encapsulating, entrapping or lyophilizing processes.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Hereinafter, the phrases “physiologically suitable carrier” and “pharmaceutically acceptable carrier” are interchangeably used and refer to an approved carrier or a diluent that does not cause significant irritation to an organism and does not abrogate the biological activity and properties of the administered conjugate.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should be suitable for the mode of administration.

Herein the term “excipient” refers to an inert substance added to a pharmaceutical composition to further facilitate processes and administration of the active ingredients. Examples, without limitation, of excipients include calcium carbonate, calcium phosphate, various sugars and types of starch, cellulose derivatives, gelatin, vegetable oils and polyethylene glycols. In one preferred embodiment, the pharmaceutically acceptable excipient is carboxymethylcellulose, microcrystalline cellulose, starch and modified starch.

Further techniques for formulation and administration of active ingredients may be found in “Remington's Pharmaceutical Sciences,” Mack Publishing Co., Easton, Pa., latest edition, which is incorporated herein by reference as if filly set forth herein.

The pharmaceutical compositions herein described may also comprise suitable solid or gel phase carriers or excipients. Examples of such carriers or excipients include, but are not limited to, calcium carbonate, calcium phosphate, various sugars, starches, cellulose derivatives, gelatin and polymers such as polyethylene glycols.

In a preferred embodiment of the present invention, the selenium unit dosage is administered orally. Other suitable routes of administration may, for example, include rectal, transmucosal and other enteral routes, transdermal, intestinal or parenteral delivery, including intramuscular, subcutaneous and intramedullary injections as well as intrathecal, direct intraventricular, intravenous, intraperitoneal, intranasal, or intraocular injections.

Pharmaceutical compositions for use in accordance with the present invention thus may be formulated in conventional manner using one or more pharmaceutically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological saline buffer. For transmucosal administration, penetrants are used in the formulation. Such penetrants are generally known in the art.

For transmucosal administration, penetrants are used in the formulation. Such penetrants are generally known in the art. For oral administration, the active ingredients can be formulated readily by combining the active ingredients with pharmaceutically acceptable carriers well known in the art. Such carriers enable the active ingredients of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added All formulations for oral administration should be in dosages suitable for the chosen route of administration. For buccal administration, the compositions may take the form of tablets or lozenges formulated in conventional manner.

The active ingredients described herein may also be formulated for parenteral administration, e.g., by bolus injection or continuous infusion.

Pharmaceutical compositions for parenteral administration include aqueous solutions of the active preparation in water-soluble form. Additionally, suspensions of the active ingredients may be prepared as appropriate oily injection suspensions. Suitable lipophilic solvents or vehicles include fatty oils such as sesame oil, or synthetic fatty acids esters such as ethyl oleate, triglycerides or liposomes. Methods for preparing liposomes loaded with selenium compounds are well known in the art (see, for example, U.S. Pat. No. 5,994,151 to Spallholz et al). Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

The active ingredients described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

In a preferred embodiment, the composition is formulated in accordance with routine procedures as a pharmaceutical composition adapted for intravenous administration to human beings. Typically, pharmaceutical compositions for intravenous administration are solutions in sterile isotonic aqueous buffer. Where necessary, the pharmaceutical composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active ingredients of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of active ingredient effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any active ingredient used in the methods of the invention, the therapeutically effective amount or dose can be estimated initially from activity assays in animals. For example, a dose can be formulated in animal models to achieve a circulating concentration range that includes the IC₅₀ as determined by activity assays. Such information can be used to more accurately determine useful doses in humans. In general, dosage is from about 0.01 micrograms to about 100 mg per kg of body weight, and may be given once or more daily, weekly, monthly or yearly.

Toxicity and therapeutic efficacy of the active ingredients described herein can be determined by standard pharmaceutical procedures in experimental animals, e.g., by determining the IC₅₀ and the LD₅₀ (lethal dose causing death in 50% of the tested animals) for a subject active ingredient. The data obtained from these activity assays and animal studies can be used in formulating a range of dosage for use in human. For example, therapeutically effective doses suitable for treatment of autoimmune, musculoskeletal, neurological, cardiovascular and other inflammatory conditions can be determined from the experiments with animal models of these diseases described hereinbelow.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma and/or tissue levels of the active moiety which are sufficient to maintain the modulating effects, termed the minimal effective concentration (MEC). The MEC will vary for each preparation, but can be estimated from in vitro data; e.g., the concentration necessary to achieve 50-90% inhibition of a heparanase may be ascertained using the assays described herein. Dosages necessary to achieve the MEC will depend on individual characteristics and route of administration. HPLC assays or bioassays can be used to determine plasma and/or tissue concentrations.

Dosage intervals can also be determined using the MEC value. Preparations should be administered using a regimen, which maintains plasma and/or tissue levels above the MEC for 10-90% of the time, preferable between 30-90% and most preferably 50-90%.

Depending on the severity and responsiveness of the condition to be treated, dosing can also be a single administration of a slow release composition described hereinabove, with course of treatment lasting from several days to several weeks or until cure is effected or diminution of the disease state is achieved.

The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

Suppositories generally contain active ingredient in the range of from about 0.5% to about 10% by weight; oral formulations preferably contain from about 10% to about 95% active ingredient.

The amount of a composition to be administered will, of course, be dependent on the subject being treated, the severity of the affliction, the manner of administration, the judgment of the prescribing physician, etc.

It is to be understood that the method and article of manufacture of the present invention will have utility for non-human subjects as well as for humans. That is, the appended claims are intended to include veterinary uses of the new method and article of manufacture according to the invention. Animals also suffer from inflammatory disorders that are similar to the inflammatory disorders recited hereinabove, and the composition of the present invention will be useful for treating such animals.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Experimental Procedures

Animals: Male Sprague-Dawley rats (approximately 150 g bw) received food and water ad libitum. They were kept in plastic cages with wire tops in a light-controlled room. All animals were cared for under the guidelines set forth by the Animal Care Committee of the Hebrew University, Jerusalem, Israel.

High-dose dietary selenium supplementation: Rats received food and (unsupplemented) water ad libitum (control group). Normal selenium (control) diet included 0.2 mg selenium/kg chow. The selenium-treated group received the same chow and 16 ppm sodium selenite-supplemented water, ad libitum for 20 days. The high-selenium diet provided approximately 2-3 μ/gm day of selenium per animal, which is considered an acceptable excess supplementation diet for this element (Davis et al., 1998; Shilo et al., 2003).

Induction of colitis and sample collection: Colitis was induced by administration of 0.5 ml of 2,4,6-trinitrobenzene sulphonic acid (TNBS, 100 mg/ml dissolved in 50% ethanol)through the anal canal, at a distance of 8 cm into the colon, just proximal to the splenic flexure. 24 hours after induction animals were sacrificed and the colon was removed for pathology, histocytological, biochemical or immunohistocytological analysis (Reifen et al., 2000).

Tissue inflammation-Myeloperoxidase activity: Colonic tissue samples (approximately 100-120 mg) were collected 4 cm proximal to the anus. Each tissue sample was homogenized in a solution containing 0.5% hexa-decyl-trimethyl-ammonium bromide (HTAB) (Sigma-Aldrich Corp, St. Louis, Mo.) buffer (0.5% HTAB in 50 mM phosphate buffer, pH 6.0). Tissue samples were minced in a test tube containing 1 ml of HTAB buffer on ice and homogenized with a Polytron tissue homogenizer (Brinkmann Instruments, Inc, Westbury N.Y., USA). The pooled homogenates and washes were sonicated in water for 20 seconds. After three cycles of freeze-thawing, the sonicated samples were centrifuged in cold for 15 min. at 40,000×g. Myeloperoxidase activity (MPO) was assayed in the supernatant as described by Krawisz (Krawisz et al., 1984) after addition of O-dianizidine-HCl (Sigma-Aldrich Corp, St Louis, Mo.) and 0.15% (v/v) H₂O₂ as a substrate for the MPO. The rate of change in absorbance was measured spectrophotometrically at 460 nm, and is expressed as the amount of enzyme necessary to produce a change in absorbance of 1.0 unit per minute (1 unit) per gram wet weight tissue.

Western blotting—analysis of Cytochrome c: Colon tissue samples were boiled and kept in sample buffer (Sigma-Aldrich Corp., St Louis, Mo.) immediately following isolation. For electrophoretic separation and immunodetection, the samples were reboiled, and subjected to SDS-PAGE followed by Western blot analysis. Briefly, separated proteins were transferred electrophoretically from the gel to a nitrocellulose membrane (Amersham International plc, Buckinghamshire, England). The membrane was blocked in TBS (0.15 M NaCl/10 mM Tris/HCl [pH7.4]) containing 5% (v/v) skim milk (Blotto), then incubated overnight with the primary antibody at 4° C. (diluted 1/1000 in Blotto). Following six washes in TBST (TBS containing 0.05% v/v Tween 20), the membrane was incubated for 2 hours at room temperature with the secondary antibody (diluted 1/1000 in Blotto). Immunoreactive bands were detected with ECL western blotting detection reagents and developed on film (Shilo et al., 2003). Separated proteins were transferred electrophoretically from the gel to a nitrocellulose membrane (Amersham International plc, Buckinghamshire, England). The membrane was blocked in TBS (0.15M NaCl/10 mM Tris/HCl (pH7.4)) containing 5% (v/v) skim milk (Blotto), then incubated overnight with the mouse anti-mouse cytochrome c primary antibody (BD Biosciences Pharmingen, San Diego, Calif.) at 4° C. (diluted 1/1000 in Blotto). After washing six times in TBST (TBS containing 0.05% v/v Tween 20), the membrane was incubated for 2 hours at room temperature with a chemiluminescent goat anti mouse IgG second antibody (BD Biosciences Pharmingen, San Diego, Calif.) (diluted 1/1000 in Blotto). Immunoreactive bands were visualized by detection with ECL Western blotting detection reagents (ECL, Amersham Biosciences, Uppsala, Sweden) followed by exposure of film and quantitation by densitometry (Shilo et al., 2003).

Mitochondrial DNA analysis in the colon: Tissue content of mitochondrial DNA was determined using specific primers for the D-loop region in the mitochondrial DNA. Mitochondrial colon tissue genomic DNA, was prepared using the Promega Wizard Genomic DNA Purification Kit (Promega Corp, Madison, Wis.). Mitochondrial DNA was amplified by the PCR reaction: Twenty cycles were used for the amplification with 25 ng total tissue DNA as template. Saturation in intensity of PCR products was observed after 24 cycles.

Primers: Left (sense) 5′-GGTTCTTACTTCAGGGGCCATC-3′ (SEQ ID NO:1), corresponding to nucleotides 468 to 489 of rat mitochondrial D-loop region DNA (GenBank Accession No. X52757.1).

Right (antisense) 5′-GTGGAATTTTCTGAGGGTAGGC-3′ (SEQ ID NO:2) corresponding to nucleotides 988 to 967 of rat mitochondrial D-loop DNA (GenBank Accession No. X52757.1).

The expected PCR product was 520 bp. Actual PCR product was approximately 520 bp, measured on 1.25% agarose gel in TAE containing ethidium bromide.

Mitochondrial (mt) TFA, NRF1, and cytochrome c mRNA analysis: Total RNA was extracted from 50 to 100 mg tissue at a time using 1 ml of Tri Reagent® (Sigma-Aldrich Corp., St. Louis, Mo.) according to manufacturer's instructions. First strand cDNA synthesis was carried out using reverse-iTTM First Strand Synthesis from Advanced Biotechnologies (ABgene®, ABgene, Epsom, U.K.) was used according to the manufacturer's instructions. Target sequences were amplified in the PCR reaction as follows: each tube contained 37 ng of cDNA from cytochrome c, GAPDH, or 18S, 3 μl of specific primers, 12.5 μl of ready Mix and 6.5 μl water DEPC (for a total 25 μl in each tube). The linear PCR response was observed at 24 cycles for GAPDH and 19 cycles for 18S.

The PCR for cytochrome c, NRF1 and mtTFA was run at 95° C. for 5 min, 94° C. for 30 sec, 56° C. for 2 min, 72° C. for 1 min, back to stage 2 for 22, 26, 24 cycles, respectively, then 72° C. for 10 min, and 4° C. The products of the PCR were separated on a 1% agarose gel in TAE buffer with ethidium bromide. The electrophoresis took place in a BioRad device in 1×TAE buffer at 95 V for about 50 min (maximum mA). The size of the cDNA was determined by a 100-bp marker.

Primers:

Rat Cytochrome c: (Antisense) 5′-GGA GGC AAG CAT AAG ACT GG-3′ (SEQ ID No. 11) corresponding to nucleotides 70 to 89 of rat cytochrome c coding sequence (GenBank Accession No. M20622).

(Sense): 5′-GTC TGC CCT TTC TCC CTT CT-3. (SEQ ID No. 12). The expected PCR product was 211 bp, the actual 211 bp.

Rat NFR-1: (Antisense) 5′-ACC TTT GGA GAA TGT GGT GC-3′ (SEQ ID No. 13) corresponding to nucleotides 450 to 469 of rat NFR-1 (GenBank Accession No. XM231566).

(Sense): 5′-GTG ATG GTA CGA GAT GGG CT -3′ (SEQ ID No. 14). The expected PCR product was 461 bp, the actual 461 bp.

Rat mtTFA: (Antisense) 5′-GGA AGA GCA AAT GGC TGA AG-3′ (SEQ ID No. 15) corresponding to nucleotides 756 to 777 of rat mtMTFA (GenBank Accession No. AJ312746).

(Sense): 5′-AGA ACT TCA CAA ACC CGC AC-3′ (SEQ ID No. 16). The expected PCR product was 417 bp, the actual 417 bp.

Rat 18S rRNA: (Antisense) 5′-CGG CTA CCA CAT CCA AGG AA-3′ (SEQ ID No. 17) corresponding to nucleotides 452 to 471 of (Embl. Accession No. X01117).

(Sense): 5′-CGC TAT TGG AGC TGG AAT TAC C-3′ (SEQ ID No. 18). The expected PCR product was 196 bp, the actual 196 bp.

Rat GAPDH: (Antisense) 5′-GCC ATC AAC GAC CCC TTC AT-3′ (SEQ ID No. 19) corresponding to nucleotides 164 to 183 of rat GADPH coding sequence (GenBank Accession No. BC059110).

(Sense): 5′-TTC ACA CCC ATC ACA AAC AT-3′ (SEQ ID No. 20). The expected PCR product was 314 bp, the actual 314 bp.

Mitochondrial respiration activity in colon tissue: Mitochondrial respiration was measured by direct assessment of oxygen consumption of the tissue. Briefly, colons were removed, chilled on ice, and washed extensively with cold PBS. Colon tissue samples (5-10 mm in width, 100 mg) were incubated at ambient temperature in PBS supplemented with 5 mM glucose. Oxygen consumption was measured polarographically using a computerized Clark-type oxygen electrode. The addition of the Electron Transport Chain (complex 1) inhibitor rotenone was used to determine whether the majority of oxygen consumption measured in the colon samples was mitochondrial respiration-dependent.

Lipid peroxidation—MDA levels: MDA levels in the colon tissue were determined as an indicator of lipid peroxidation using HPLC methodology (Valacchi et al., 2000). Briefly, tissue sections (100 mg) were homogenized in 1% Triton X 100 PBS. The suspended tissue was diluted 1:1 with 1% SDS solution and incubated with 250 ul TBA reagent (0.375 g TBA/100 ml) and 200 ul phosphoric acid (1.22 M) for 30 min at 100° C. Methanol (380 ml) was added for protein precipitation and NaOH (20 ml, 1N) added for pH neutralization. After centrifugation, 100 ul clear supernatant was injected into a HPLC system, using a C185 m, 250 mm^{a} 4.6 mm internal diameter, reversed phase column (Alltech, Deerfield, Ill., USA). The mobile phase consisted of 60% methanol and 40% 50 mM NaH₂PO₄, pH 5.5. The flow rate was 0.9 ml/min and the detector was set to 532 nm. HPLC colarray ESA was used.

Measurement of selenium incorporation into colon tissue: inductively coupled plasma (ICP) method: Colon tissue samples were prepared for analysis of selenium content by microwave-assisted digestion using an MLS 1200 mega microwave digestion unit [Milestone Sorisole (BG), Italy]. At the conclusion of the digestion period the vessels were allowed to cool down to room temperature and were uncapped. Liquid residues were taken up in water, transferred into 25-ml calibrated flasks and equilibrated to 25 ml volume with water. Analyses were conducted on portions of these solutions, versus multi-element selenium standards from Merck (Merck and Co, EMBD Biosciences, San Diego Calif.) in the same solvent. Selenium (Se) was determined by ICP-atomic emission spectrometry (AES) at 196.090 nm. An ICP-AES system, model “Spectroflame Modula E” from Spectro, Kleve, Germany was used with a cross-flow nebulizer. The power level was 1.2 kW, coolant flow 15 l/min, auxiliary flow 0.5 l/min and nebulizer flow 0.5 l/min. Observation height was 10 mm above the coil.

Cell culture: Murine J774.2 macrophages (kindly provided by Prof. Dov Zipori from the Weizmann Institute of Science) were grown in RPMI medium supplemented with 10% (w/v) fetal calf serum, penicillin (100 U/ml), streptomycin (100 μg/ml) and 1× glutamine at 37° C. in a humidified atmosphere consisting of 95% air and 5% CO₂. Experiments were performed on cells at 60% confluence.

Determination of cell viability: Cell-membrane integrity was detected by flow cytometer (FACSort, Becton Dickenson and Co., San Jose, Calif. USA) as a measurement of cell viability. For this assay, the nonpermeant DNA-intercalating dye propidium iodide (PI), which is excluded by viable cells, was used. Fluorescence settings were excitation at 488 nm and emission at 575 nm. Data were collected from at least 10,000 cells.

Determination of type of cell death: Type of cell death was determined by dual staining with annexin V-FITC (Santa Cruz Biotechnology, Santa Cruz Calif., USA) and PI, and flow cytometric analysis. Cells were treated with 10 μM selenite for 24 h then collected and re-suspended in binding buffer (10 mM HEPES/NaOH, pH 7.4, 140 mM NaCl, 2.5 mM CaCl2). 2.5 μg/ml annexin V-FITC was added and cells were incubated for 30 min at room temperature, then 0.25 μg/ml PI was added and samples were analyzed by flow cytometry (FACSort, Becton Dickenson and. Co., San Jose, Calif. USA).

Intracellular Radical Oxygen Species (ROS) (H₂O₂): Intracellular ROS were detected using dihydrodichlorofluorescein (H₂DCF-DA). Cells exposed to different treatments were washed three times with PBS. Cells were centrifuged (2000 RPM, 5 min), resuspended in PBS and incubated with 25 μM H2DCF-DA for 30 min at 37° C. To detect intracellular fluorescence, the fluorochrome-loaded cells were excited using a 488-nm argon-ion laser in a flow cytometer. The dichlorofluorescein (DCF) emission was recorded at 530 nm. Data were collected from at least 10,000 cells. Alternatively, DCF fluorescence was recorded using a microfluorometer plate reader (GENios, Tecan, Austria). Cells were washed twice with PBS, then 25 μM H2DCF-DA was added and cells incubated for 30 min at 37° C. Following an additional wash, cells were lysed with PBS-0.2% Triton-X 100. Total fluorescence was recorded and calibrated according to protein content.

Western blots (Sp1, MnSOD, UCP2): Cells were grown in six-well tissue culture plates, treated with selenium for 72 h and activated with LPS and IFN-γ for 24 h. The wells were washed with PBS, scraped and centrifuged

(5 min. 2000 RPM), and lysed by addition of boiling hot lysis buffer (1% SDS, 1 mM sodium ortho-vanadate, 10 mM Tris pH 7.4) to the pellets. Protein content was measured (BIO RAD protein assay kit) and equalized. Sample buffer (containg SDS) was added and the samples were boiled. The samples were subjected to SDS-PAGE followed by western blot analysis. Separated proteins were transferred electrophoretically from the gel to a nitrocellulose membrane (Amersham International plc, Buckinghamshire, ENGLAND). The membrane was blocked in TBS (0.15 M NaCl/10 mM Tris/HCl, pH7.4) containing 5% (v/v) skimmed milk (Blotto), then incubated over night with primary antibodies at RT (diluted 1/2000 in blotto). After being washed six times in TBST [TBS containing 0.05% (v/v) Tween 20], the membrane was incubated for 2 h at room temperature with secondary antibody (diluted 1/5000 in blotto). Immunoreactive bands were detected with ECL western blotting detection reagents (Amersham Pharmacia Biotech, Buckingammshire England) and developed on film (Fuji super RX, Dusseldorf, Germany). Primary antibodies for Sp1 and MnSOD were from BD Transduction Laboratories (Becton Dickenson and Co., San Jose, Calif. USA) and for UCP2 from Alpha Diagnostics LTD (Warszaw, Poland). Secondary antibodies were from Jackson ImmunoResearch (Jackson Labs, Bar Harbor, Maine, USA).

Nuclear extracts: Nuclear Extracts were prepared using a slight modification of the method of Dignam et al (23). Treated cells were washed with cold PBS, scraped and centrifuged. Hypotonic buffer (1 ml, 20 mM HEPES pH 7.0, 10 mM KCl, 1 mM mGCl2, 0.5 mM DTT, 0.1% Triton X-100, 20% glycerol, 2 mM PMSP, 1 mg/ml Aprotonin and Leupeptin) was added to the cell pellets, the pellets were disrupted by pipetting, and centrifuged 3000 RPM for 5 min. The pellets were re-suspended in 100 μl cold extraction buffer (20 mM HEPES pH 7.0, 10 mM KCl, 1 mM mGCl2, 0.5 mM DTT, 0.1% Triton X-100, 20% glycerol, 2 mM PMSF, 1 mg/ml Aprotonin and Leupeptin, 420 mM NaCl). The samples were rotated for 20 min at 4° C., centrifuged 15,000 RPM for 10 min, and supernatants collected. Protein content was measured using Bradford reagent. Samples were stored frozen at minus 70° C.

Electromobility shift assay (EMSA)—Gel shift assays were performed according to the gel shift assay protocol from Promega Corp. (San Luis Obispo, Calif.). A 22-mer double-stranded NF-κB consensus oligonucleotide 5′-AGTTGAGGGGACTTTCCCAGGC-3′ (SEQ ID NO:3), and Sp1 consensus oligonucleotide 5′-ATTCGATCGGGGCGGGGCGAGC-3(SEQ ID NO:4), were labeled with 32P. For each reaction, 2 μg of nuclear extract were incubated with 2 μl of 5× binding buffer for 5 min., 1 μl of the ³²P-labeled oligonucleotide was added at room temperature for 20 min., the samples were prepared by adding 1 μl of dye free loading buffer, and separated using 6% non-denaturing acrylamide gel electrophoresis. The gel was dried and exposed to a phosphoimaging screen for 18 hours and then visualized with a phosphoimager. For specificity, 1 μl of unlabeled consensus oligonucleotide was used to compete with the labeled probe. For in vitro experiments, 5 μM of sodium selenite was added to the nuclear extracts prior to addition of the binding buffer, and incubated for 5 min.

Evaluation of mitochondrial membrane potential (MMP) in cells—MMP was detected by flow cytometry (FACSort, Becton Dickenson and Co., San Jose, Calif. USA) using the membrane potential-sensitive fluorescent probe MitoTracker Orange (CMTMRos) (Molecular Probes, Oregon, USA). The following fluorescence setting was used: excitation at 488 nm and emission at 575 nm (FL2 channel) (24,25). Macrophage cells (1 million cells/ml) were stained with 0.1 μM CMTMRos for 30 min at 37° C. Accumulation of the dye in the mitochondria was evaluated by flow cytometer analysis as described hereinabove. Data were collected from 10,000 cells.

GPx activity: GPx activity was determined by following the rate of NADPH oxidation at 340 nm in the presence of the substrates: 3 mM glutathione (GSH), 1.2 U glutathione reductase (GR) and 1.5% H₂O₂, using a microfluorometer plate reader (GENios, Tecan, Austria). The reaction contained 500 μl Tris reaction buffer (100 mM Tris Hcl, 300 mM Kcl, 5 mM EDTA, 1 mM NaN3, pH 7.0), substrates and 100 μl cell extract. Macrophages were treated with selenium for 24 h. Wells were washed twice with PBS, cells permeabilized, and scraped using 200 μl 0.2% Triton X-100. The reaction was recorded every minute for 25 minutes. Slopes were calculated and the results were adjusted according to the protein amount calculated with the Bradford reagent.

Reverse transcriptase-PCR (RTPCR): Total RNA was prepared and isolated by the Tri-Reagent method according to the manufacturer's protocol (Sigma, St. Louis Mo., USA). Total RNA (1 μg) was converted into cDNA using Reverse-iT first strand synthesis kit (Abgene, UK). Amplification of MnSOD and UCP2 sequences was performed by incubating 10 ng equivalents of DNA with the following oligonucleotide primers:

For detection of MnSOD:

Left (sense) 5′-ATGTTGTGTCGGGCGGCG-3′(SEQ ID NO:5) corresponding to nucleotides 69 to 87 of mouse mRNA region (GenBank Accession No. NM 013671).

Right (antisense) 5′AGGTAGTAAGCGTGCTCCCACACG-3′ (SEQ ID NO: 6) corresponding to nucleotides 622 to 640 of mouse mRNA region (GenBank Accession No. NM 013671). The expected PCR product was 571 bp. Actual PCR product was approximately 570 bp, measured on 1.25% agarose gel in TAE containing ethidium bromide.

For detection of UCP2:

Left (sense), 5′-TTCAAGGCCACAGATGTGCC-3′ (SEQ ID NO:7) corresponding to nucleotides 372 to 392 of mouse mRNA (GenBank Accession No. NM 011671).

Right (antisense) 5′TCGGGCAATGGTCTTGTAGGC-3′ (SEQ ID NO: 8) corresponding to nucleotides 826 to 846 of mouse mRNA (GenBank Accession No. NM 011671). The expected PCR product was 474 bp. Actual PCR product was approximately 474 bp, measured on 1.25% agarose gel in TAE containing ethidium bromide.

The sequence of glyceraldehyde-3-phosphate dehydrogenase (GAPDH) was amplified as a control using the following primers: Left (sense) 5′-TCCGCCCCTTCCGCTGATG-3′(SEQ ID NO: 9) corresponding to nucleotides 364 to 382 of mouse mRNA (GenBank Accession No. XM 195823.21).

Right (antisense) 5′CACGGAAGGCCATGCCAGTGA-3′ (SEQ ID NO: 10) corresponding to nucleotides 683 to 703 of mouse mRNA (GenBank Accession No. XM 195823.21). The expected PCR product was 339 bp. Actual PCR product was approximately 340 bp, measured on 1.25% agarose gel in TAE containing ethidium bromide.

PCR was performed with 28 cycles (25 cycles for GAPDH) of 30 seconds at 96° C., 30 seconds at 58° C. and 1 min at 72° C. RTPCR products were analyzed on 1.3% (w/v) agarose gels.

Statistical analysis: Comparisons between two groups were performed by t-test For multiple groups, data were analyzed by ANOVA. Differences were considered significant at probability levels of P<0.05 using the Fisher's protected least significant difference method. Statistical analysis was performed using statistical computer program, SPSS version 8 (SPSS Inc. Chicago, Ill., USA).

### EXPERIMENTAL RESULTS

### Example I

### High-dose Dietary Selenium Supplementation Prevents Hapten-induced Colon Inflammation

Dietary selenium supplementation has been recommended for a variety of conditions, including inflammatory disorders, tumor growth and metastasis, cancerous conditions, immune dysfunction, dysregulation of the cell-cycle and apoptosis, parasitic and viral infection, heart disease, reproductive and fertility disorders and even adverse mood states. However, supplementation is typically directed to prevention or correction of a perceived dietary selenium deficiency, based on official Recommended Daily Allowance (RDA) levels. Little information on the effect of tissue selenium enrichment on inflammatory and other conditions is available.

In order to investigate the efficacy of short-term, high-dose dietary selenium supplementation on inflammatory conditions, experimental colitis was induced by hapten enema in rats receiving three week's high dose selenium supplementation, supplied via drinking water.

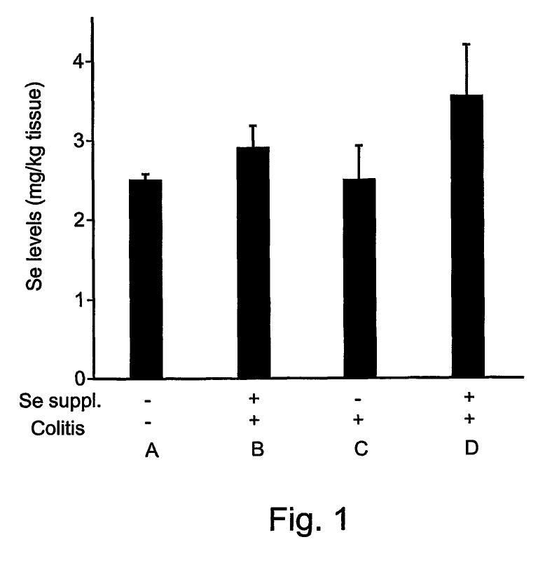

Selenium tissue enrichment with high-dose dietary selenium supplementation: It has been shown that blood selenium concentrations are correlated with the dietary intake, and that selenium supplementation, in the form of low dose, commercially available supplements, increases blood selenium concentrations accordingly (Schrauzer G N and White D A, Bioinorg Chem 1978;8:303-18). However, tissue selenium levels were not reported. Sensitive measurement of tissue selenium levels in healthy and colotis-induced rats indicated that high doses of dietary selenium are required to effect significant elevation of tissue selenium levels in the colon. Three weeks of high-dose dietary selenium supplementation (16 ppm sodium selenite in the water), equivalent to 0.16-0.25 μg/g per day, or approximately 100 times the amount considered a selenium sufficient dietary intake in animals, resulted in an increased tissue selenium content of approximately 20% (FIG. 1, column A vs. column B) in healthy rats. The increase in tissue selenium levels was greater in colitis-induced rats (FIG. 1, columns C and D), although little change in colon tissue selenium levels was observed in the unsupplemented colitis-induced group, indicating that hapten-induced colitis has little direct effect on the tissue selenium levels. Thus, short-term, high dose dietary selenium supplementation, in the range of about 5-13000 μg/kg per day, according to the methods of the present invention, is effective in significantly increasing tissue selenium levels in mammals.

High dose dietary selenium supplementation prevents inflammation and necrosis in hapten-induced colitis: The hapten (TNBS)-induced colitis model of Inflammatory Bowel Disease (IBD) has been shown to produce chronic colitis, with the characteristic imbalance of regulatory cytokines, severe inflammation of the mucosal layer, and delayed type hypersensitivity to TNP-modified self antigens (Dohi et al, J Exp Med 1999;189:1169-80). In order to determine whether the increased tissue selenium levels resulting from high dose selenium supplementation are effective in preventing hapten-induced inflammation of the colon, gross pathological and biochemical analysis was performed on colon tissue from healthy and hapten-induced rats receiving high dose dietary selenium supplementation and from unsupplemented controls.

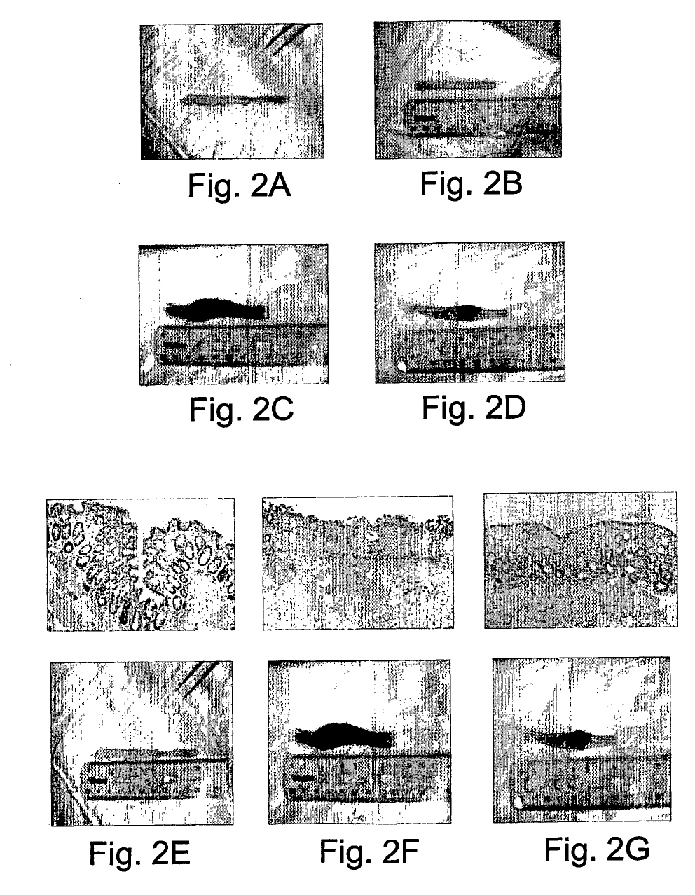

FIGS. 2C and 2D show a representative gross pathological analysis of colon tissue resected 24 hours after administration of TNBS, as described in Materials and Experimental Methods hereinabove. Characteristic discoloration of necrotic tissue is evident over more than 80% of the colon from the unsupplemented, hapten-induced rat (FIG. 2C), whereas necrosis is clearly greatly reduced, limited to less than 15%, in the colon from a rat after 3 weeks high-dose dietary selenium supplementation (FIG. 2D). Examination of tissue from healthy, uninduced selenium-supplemented (FIG. 2B), and uninduced, unsupplemented control rats (FIG. 2A) revealed no apparent effects of the high-dose dietary selenium supplementation on the colon tissue.

Microscopic analysis of the changes in colon tissue histology revealed mucosal necrosis with fresh hemorrhaging following hapten induction of inflammation (FIGS. 2E-2F), with heavy, diffuse neutrophilic infiltrate of the muscularis externa, and perivascular and vascular neutrophilic infiltration with evidence of vascular necrosis within areas of mucosal necrosis. Analysis of colon tissue from a rat receiving 3 weeks high-dose dietary selenium supplementation (FIG. 2G) revealed preservation of the mucosal architecture, minimal foci of attenuation and erosion of the mucosal epithelium, and mild proprial edema, with normal appearance of the vasculature. Thus, high-dose dietary selenium supplementation, unexpectedly protected rat colon tissue from hapten-induced inflammation-related necrosis.

Inflammation is characterized by an infiltration and activation of neutrophils, resulting in an oxidative burst producing highly elevated myeloperoxidase activity. Myeloperoxidase activity, and the reactive oxygen species resulting therefrom, have been implicated in the pathogenesis of many inflammatory diseases (see, for example, Spickett, C M et al, Acta Biochim Pol 2000; 47:889-99). In order to determine whether high-dose dietary selenium supplementation can prevent inflammatory processes associated with hapten-induced colitis, myeloperoxidase activity was measured in high dose dietary selenium supplemented, and unsupplemented control rat colon tissue following administration of hapten (TNBS) enema.

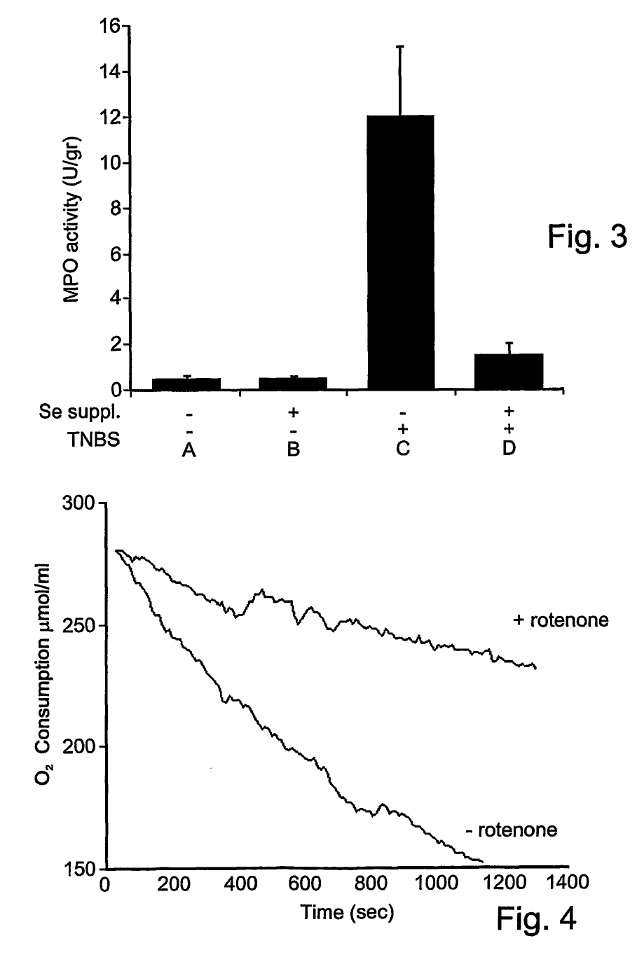

Myeloperoxidase activity, measured spectrophotometrically in cell-free supernatants of homogenized colon tissue samples prepared as described hereinabove, was elevated greater than 30-fold 24 hours after hapten induction of colitis in the unsupplemented rats, compared with un-induced controls (FIG. 3, column A compared to column C), indicating a strong inflammatory reaction to hapten (TNBS) administration. Myeloperoxidase levels in colon tissue from rats receiving high-dose dietary selenium supplementation was only mildly elevated (approximately 5 fold, compared to uninduced controls) following hapten administration (FIG. 3, column B compared to column D), demonstrating significant protection from the inflammatory effects of colitis induction.

Thus, these results show, for the first time, that high-dose dietary selenium supplementation, in the range of about 5-13000 μg/kg per day, administered over a period of weeks, can effectively prevent both the necrosis and inflammation associated with colitis in IBD.

### Example II

### High-dose Dietary Selenium Supplementation Protects Against Mitochondrial Disruption in Hapten-induced Colon Inflammation

Selenium is a component of a number of important anti-oxidant enzymes, such as glutathione peroxidase and thioredoxin reductase (Arner and Holmgren, Eur J Biochem 2000; 267:6102-09; and Brigelius-Flohe et al Atherosclerosis 2000;152:307-16); therapeutic administration of selenium in dietary or topical formulas has been associated with selenium's anti-oxidant activity (see, for example, U.S. Pat. No. 6,468,987 to Demichele, et al.; U.S. Pat. No. 5,444,054 to Garleb, et al.; U.S. Pat. No. 6,214,373 to Snowden; and U.S. Pat. Nos. 6,242,010, and 6,303,651 to Hersch, which are incorporated herein by reference as if fully set forth herein). In order to determine whether the protective effects of high-dose dietary selenium supplementation on necrosis and inflammation associated with colitis in IBD, as described in Example I hereinabove, are related to selenium's enhancement of antioxidant defense in the colon, oxidative stress and mitochondrial function following administration of hapten (TNBS) were evaluated in selenium supplemented and unsupplemented rats.

Hapten-induced colids is not mediated by oxidative stress: Oxidative stress, causing enhanced lipid peroxidation, and disruption of mitochondrial respiratory chain enzymes, glyceraldehyde-3-phosphate dehydrogenase, membrane sodium/potassium ATPase activity and membrane sodium channels, is an important factor in the pathogenesis of inflammation, shock, and ischemia/reperfusion injury (Cuzzocrea S et al., Pharmacol Rev 2001;53:175-59). Lipid peroxidation, measured according to the level of malondialdehyde (MDA), is a reliable indicator of oxidative stress (Valacchi, G et al FEBS Letters 2000;466:165-8). When MDA content in colon tissue samples from colitis-induced rats and uninduced controls was measured by HPLC analysis, no changes in colon tissue lipid peroxidation in either the selenium supplemented or unsupplemented control groups was detected (data not presented), indicating that the protective properties of selenium are independent of the micronutrient's anti-oxidant effects.

Short-term high dose dietary selenium supplementation protects against mitochondrial dysfunction and disruption in hapten (TNBS)-induced colon inflammation: Mitochondria is known to regulate cell viability as well as necrotic and apoptotic cell death. A number of inter-related mitochondrial pathways have been associated with the control of cell death: 1) Mitochondrial permeability transition (MPT) and the release of apoptotic cell death promoting factors; 2) cytochrome c release by pro-apoptotic cell death promoting factors; 3) disruption of oxidative phosphorylation and ATP production; and 4) alteration of the redox status of the cell, and over-production of reactive oxygen species (Green, D. R., and Reed, J. Cell Science, 1998;281:1309-12; Hirsch, T., et al Cell Biol Toxicol,1998; 14:141-5; Korsmeyer, S. J. et al, Cell Death Differ;2000, 7, 1166-73; Kroemer, G. et al Annu Rev Physiol, 1998;60:619-42, and Kroemer, G., et al, Immunology Today, 1997;18:4451). In order to determine whether high-dose dietary selenium supplementation affects mitochondrial integrity and function in hapten-induced colon inflammation, the effect of high-dose dietary selenium supplementation on cellular respiration and mitochondrial biogenesis in healthy and hapten-induced inflamed colon tissue were evaluated.

The mitochondria is typically responsible for cellular respiration in most tissues. In order to confirm this critical role for colon tissue mitochondria as well, respiration (as oxygen consumption) was evaluated polarographically in healthy rat colon tissue samples in the presence and absence of the electron transport chain (complex 1) inhibitor rotenone, as described hereinabove. As can be seen in FIG. 4, respiration (O₂ consumption) is strongly inhibited in the presence of 20 μg/ml rotenone, confirming that respiration in colon tissue is indeed mitochondria-dependent.

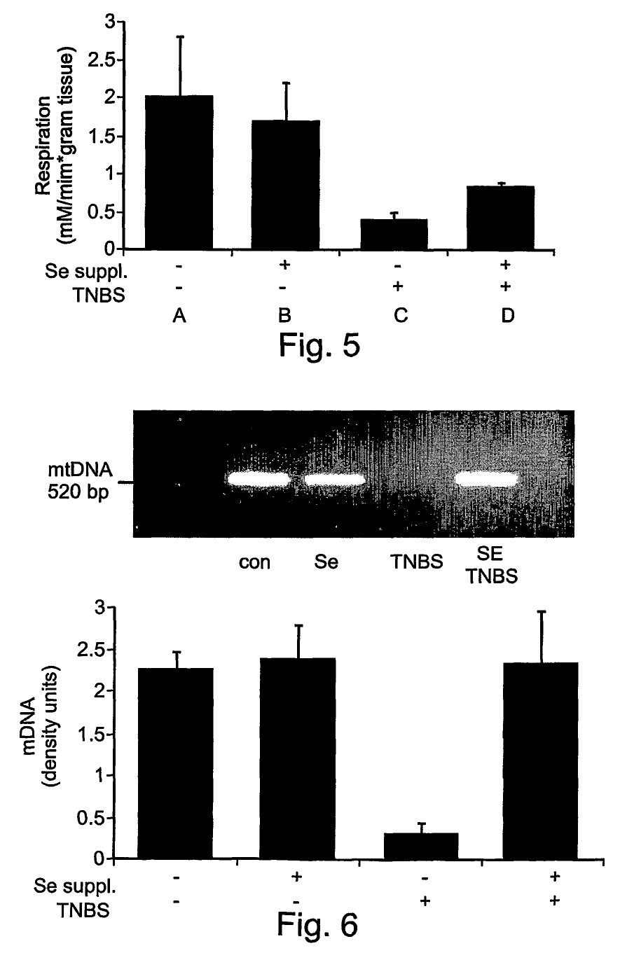

In order to evaluate the effects of high dose dietary selenium supplementation on mitochondrial function, cellular respiration was measured in colon tissue samples following selenium supplementation in healthy and hapten-induced inflammation. As can be seen from FIG. 5, high-dose dietary selenium supplementation (FIG. 5, column B) has little effect on the oxygen consumption in healthy colon tissue, compared to healthy, unsupplemented controls (FIG. 5, column A). Hapten induction of colon inflammation inhibited mitochondrial function, causing approximately 70% impairment in oxygen consumption in colon tissue from unsupplemented rats (FIG. 5, column A vs. column C). However, high dose dietary selenium supplementation surprisingly prevented a significant proportion of the hapten-induced impairment in oxygen consumption (FIG. 5, column C vs. column D).

Impairment of cellular respiration can result from disruption of mitochondrial integrity, and dysfunctional mitochondrial biogenesis. Such dysregulation of mitochondrial function has recently been linked to cell death and apoptosis, associated with cytochrome c (Liu et al, Cell 1996;86:147-57; Yang et al Science 1997;275:1129-32) and reactive oxygen species, and characterized by electron transport chain disruption and mitochondrial DNA fragmentation [Kluck et al Science 1997;275:1132-36, Mirabella et al Neurology 1996;46 (2 Suppl):A270]. In order to evaluate the effects of high dose dietary selenium supplementation on mitochondrial biogenesis, mitochondrial DNA and changes in cytochrome c protein were measured in colon tissue samples following selenium supplementation in healthy and hapten-induced inflammation.

Loss of mitochondrial DNA was observed in colon tissue samples 24 hours after induction of colitis by TNBS administration (FIG. 6, TNBS+, Se suppl−). High dose dietary selenium supplementation, however, protected the colonic tissue from such disruption of mitochondrial integrity. Colon tissue samples from colitis-induced rats receiving high dose dietary selenium supplementation had intact mitochondrial DNA (FIG. 6, TNBS+, Se suppl.−, compared to TNBS+, Se suppl+). No change in mitochondrial DNA was observed in colon tissue samples from uninduced rats receiving high dose dietary selenium supplementation FIG. 6, TNBS−, Se suppl+).

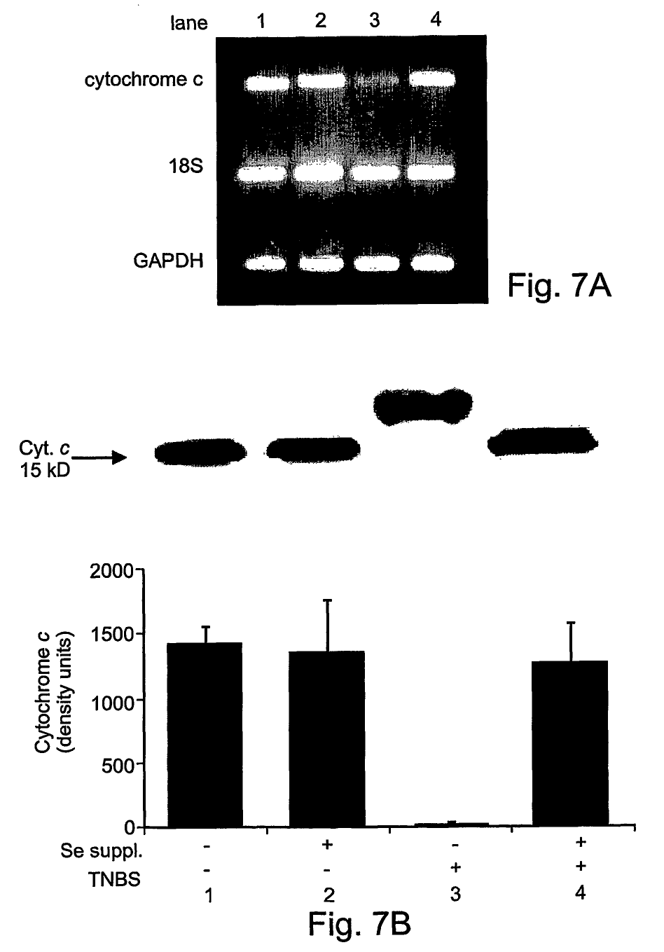

Surprisingly, high dose dietary selenium supplementation also prevented loss of cytochrome c mRNA, and the accumulation of abnormal cytochrome c in colon tissue following hapten-induced colon inflammation. RT-PCR analysis of the cytochrome c mRNA from healthy (FIG. 7A, lanes 1 and 2) and TNBS-treated (FIG. 7A, lanes 3 and 4) colon tissue clearly shows that the hapten-induced colitis specifically attenuates cytochrome c levels, without effecting tissue levels of other (18S and GAPDH RNA) transcripts (FIG. 7A, lane 3 compared to lane 1). High dose dietary selenium supplementation completely prevented the deleterious effect of TNBS-induced colitis on cytochrome c mRNA levels (FIG. 7A, lane 4), without effecting 18S or GAPDH transcription. As can be seen in the Western blot and analysis shown in FIG. 7B, all of the cytochrome c from colon tissue samples from unsupplemented rats has been converted to a form migrating more slowly upon electrophoresis following TNBS administration (FIG. 7B, lane 1 vs. lane 3). The size of this inflammation-associated slowly migrating band is clearly abnormal. However, the inflammation-associated alterations of cytochrome c were completely prevented by selenium supplementation. Extracts of colon tissue samples from hapten-induced rats receiving high dose dietary selenium supplementation showed cytochrome c protein identical to the 15 kDa band characteristic of healthy, unsupplemented controls (FIG. 7B, lane 4 compared to lanes 3 and 1). Selenium supplementation to healthy, uninduced rats had no effect on the characteristics of cytochrome c protein in colon tissue (FIG. 7B, lane 1 vs. lane 2).

Thus, short term, high dose dietary selenium supplementation in the range of about 5-13000 μg/kg per day, according to the methods of the present invention, is effective in preventing respiratory arrest, mitochondrial damage and accumulation of abnormal cytochrome c protein associated with colitis in hapten-induced colon inflammation in rats. Further, the protective effects of high dose, dietary selenium supplementation on hapten-induced colitis are independent of, or occur in addition to, any anti-oxidant effects of dietary selenium supplementation.

Studies of inflammatory bowel disease (IBD), and other inflammatory diseases indicate a central role for specific and selective alterations in mitochondrial integrity and function in the pathogenesis of IBD and the like. In patients with IBD, complex 1 (mitochondrial transport chain) activity is lower than that observed in healthy subjects. In another study, rectal biopsy specimens from control subjects, and from patients with nonrectal Crohn's and acute ulcerative colitis showed evidence of mitochondrial damage. In TNBS-treated rats, expression of cytochrome c, a nucleus-encoded protein that has to be imported into the mitochondria, was dramatically downregulated in the colitic colon. The results described herein clearly show that hapten-induced colon inflammation is characterized by breakdown of mitochondrial function and integrity. Without wishing to be limited by a single hypothesis, the protective effects of high dose, dietary selenium supplementation, preventing necrosis and regulating inflammation in the hapten-induced colon tissue, may derive from the effects of elevated tissue levels of selenium, and the micronutrient's interaction with mitochondria in inflamed and irritated tissue, distinct from selenium's anti-oxidant properties. Treatment incorporating enrichment of target tissues with high dose supplementation of selenium, provided in an easily administered, inexpensive suitable unit dosage, can be used to control the pathology of IBD, and mitochondrial damage, on a long term basis.

### Example III

### Selenium Attenuates Inflammatory Reaction in Immune Cells

In order to determine the contribution of selenium's effect on inflammatory cells in the protective effects of high dose, dietary selenium supplementation preventing of necrosis in inflamed tissue, the effect of sodium selenite on the cascade of reactive oxygen species (ROS) production in macrophages was studied in vitro in the J774.2 macrophage cell line. J774.2 macrophage cells are a murine inflammatory cell line derived from reticulum cell sarcoma, known to respond to cytokine and endotoxin stimulation.

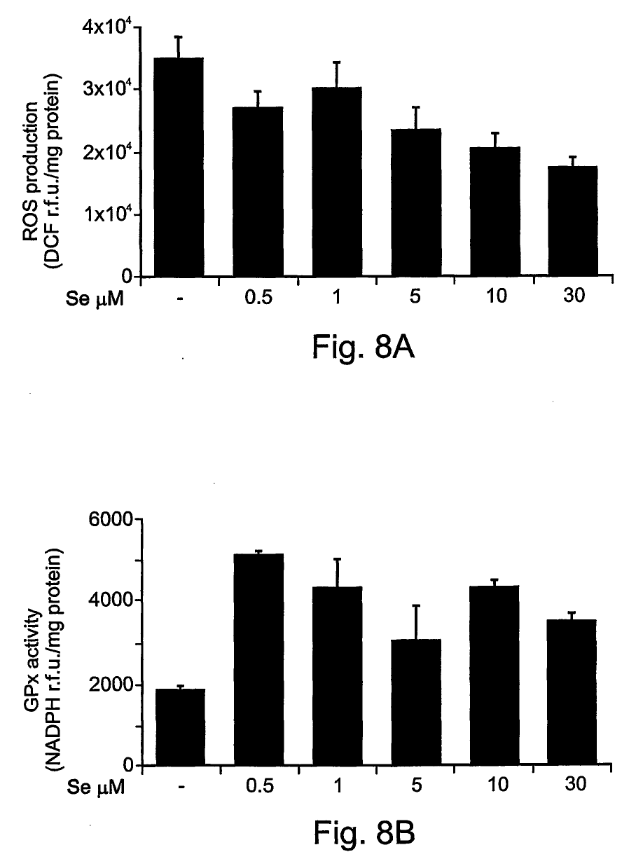

Selenium reduces macrophage intracellular peroxide production, MnSOD and UCP2 activity: While reducing the present invention to practice, it was observed that exposing J774.2 macrophage cells to elevated concentrations of selenium (0.5-30 μM) resulted in a dose-dependent decrease (>30% at 30 μM) in peroxide production, as measured by DCF fluorescence (FIG. 8A). Since the seleno-enzyme glutathione peroxidase (GPx) oxidizes glutathione, thus playing a crucial role in eliminating peroxides, the effect of selenium on GPx activity was also assayed and compared with the concentration effects on peroxide production.

As can be seen in FIG. 8B, increased activity of the enzyme GPx due to selenium exposure was maximal at 0.5 μM selenium. Without wishing to be limited to a single hypothesis, one interpretation of these results could be that the control cells were selenium-deficient as evidenced by low GPx activity, and exposure to low concentrations of selenium corrected this defficiency. Since exposure to selenium in concentrations from 0.5 to 30 μM reduced ROS production in a dose-dependent manner, additional mechanisms underlying the micronutrient's effect on macrophage inflammatory activity were sought.

The mitochondrial proteins Manganase SuperOxide Dismutase (MnSOD) and UnCoupling Protein 2 (UCP2) are crucial to the maintenance of redox balance in the cell. MnSOD is the enzyme responsible for the dismutation of superoxide radicals to peroxide in the mitochondria, and it's partial absence in partial knockout mice causes a phenotype characterized by increased sensitivity of the cells to apoptosis. UCP2 belongs to a family of mitochondnal inner membrane proteins acting as protein channels or transporters, and is strongly expressed in macrophages. In knockout experiments, it has been shown that UCP2 deficient macrophages generate higher levels of reactive oxygen species. The effect of selenium supplementation on the expression and activity levels of these mitochondrial proteins was assayed in J774.2 macrophages.

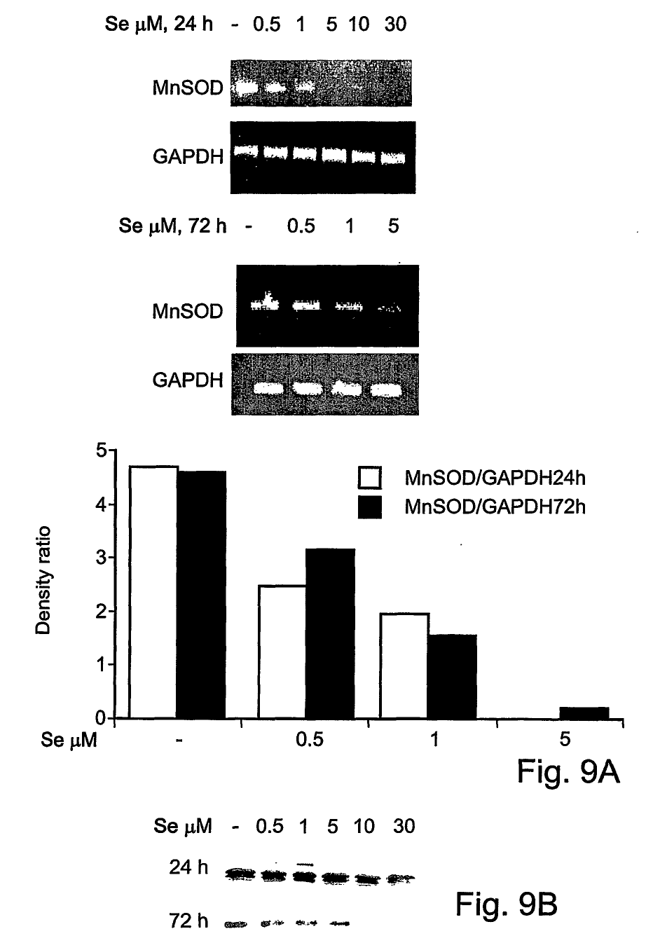

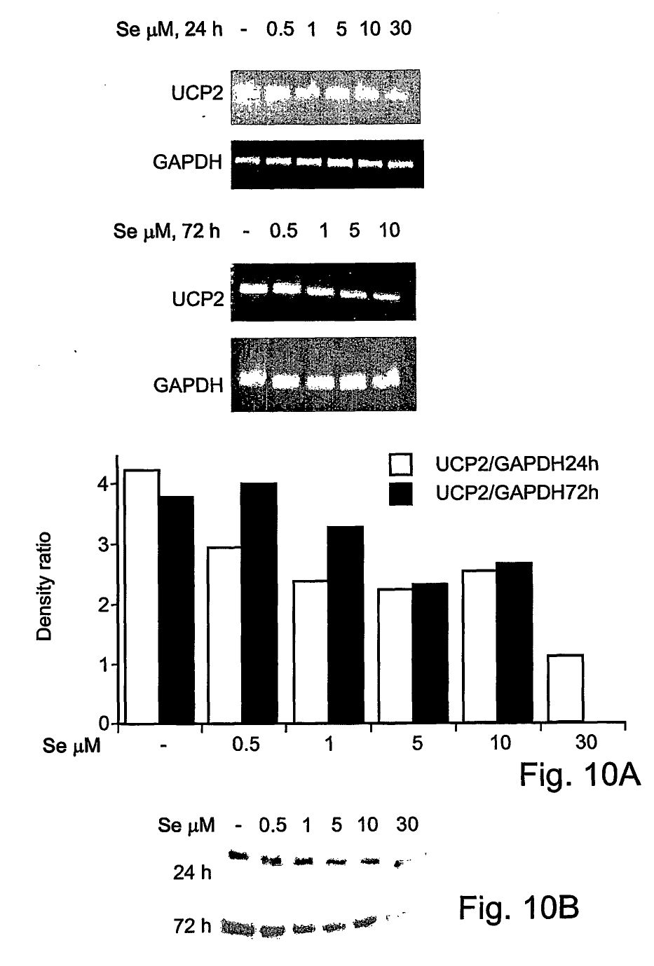

As seen in FIGS. 9A, 9B, 10A and 10B, exposure to selenium downregulated MnSOD transcription and expression, in a selective and concentration-dependent manner (FIGS. 9A and 9B). As little as 0.5 μM selenium was effective in significantly reducing the abundance of MnSOD transcripts FIG. 9A) and protein (FIG. 9B) at 24 and 72 hours. 24 or 72 hours treatment with similar concentrations of selenium also reduced the abundance of UCP2 transcripts (FIG. 10A) and protein (FIG. 10B) in a concentration dependent manner, although to a lesser extent than that of MnSOD. No effect of selenium exposure on the tissue levels of GADPH RNA was noted throughout the entire experiment (FIGS. 9A, 9B, 10A, 10B), indicating a selective effect on the MnSOD and UCP2 expression. A similar effect of selenium on MnSOD and UCP2 gene expression was observed in Jurkat T cell lymphocytes, although these cells required higher concentrations of selenium (data not shown).

Thus, these results indicate that selenium alone can significantly downregulate key enzymes in the radical oxygen species production pathway.

Selenium downregulates MnSOD and UCP2 expression in activated macrophages: Macrophage maturation plays a central role in the immune response, and causes activation of over 100 genes that encode mediators of inflammatory and immune responses, such as NF-κB, cytokines and other Stress Responsive Transcription Factors. Thus, the effect of selenium on ROS production and enzymes of the ROS production pathway was evaluated in J774.2 macrophages activated by exposure to an endotoxin (LPS) and a cytoline (INFγ).

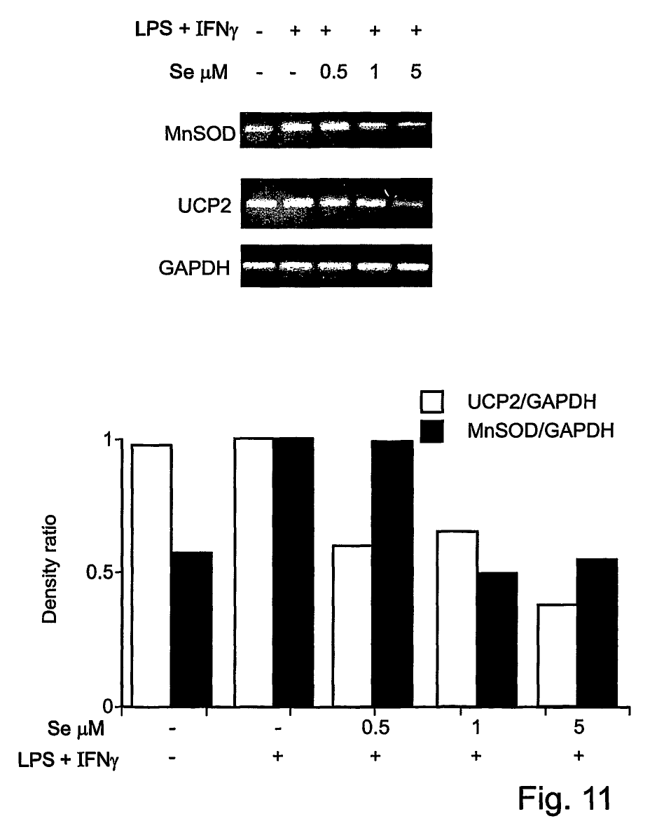

FIG. 11 shows the effect of selenium on expression of MnSOD and UCP2 in activated macrophages. Activation of control macrophages (LPS+ INFγ+, 24 hours, selenium-) upregulated MnSOD expression (FIG. 11, top panel), and had little effect on UCP2 expression. Exposure (72 hours) to selenium prior to activation of the macrophages decreased the expression of MnSOD and UCP2 expression, in a concentration dependent manner (see graph, FIG. 11). No effect of selenium on the expression of GADPH in the activated macrophages was observed, indicating no general effect of selenium on RNA transcription in the cells (FIG. 11, upper panel and graph).

Thus, selenium treatment of activated macrophages selectively reduces UCP2 expression and attenuates the overexpression of MnSOD. Focusing on these two proteins, a common denominator regulating their expression was sought. The transcription of both MnSOD and UCP2 is regulated by the ubiquitous transcription factor Sp-1, which binds GC-rich sites on DNA. In addition, activation of macrophages with LPS and INFγ is known to induce translocation of the cytoprotective nuclear factor NF-κB to the nucleus, which in turn induces the transcriptional activation of many genes encoding mediators of inflammatory and immune response. To determine whether Sp-1 and/or NF-κB are involved in downregulation of the inflammatory response in macrophages, the effect of selenium treatment on Sp-1 and NF-κB binding in activated J774.2 macrophages was investigated.

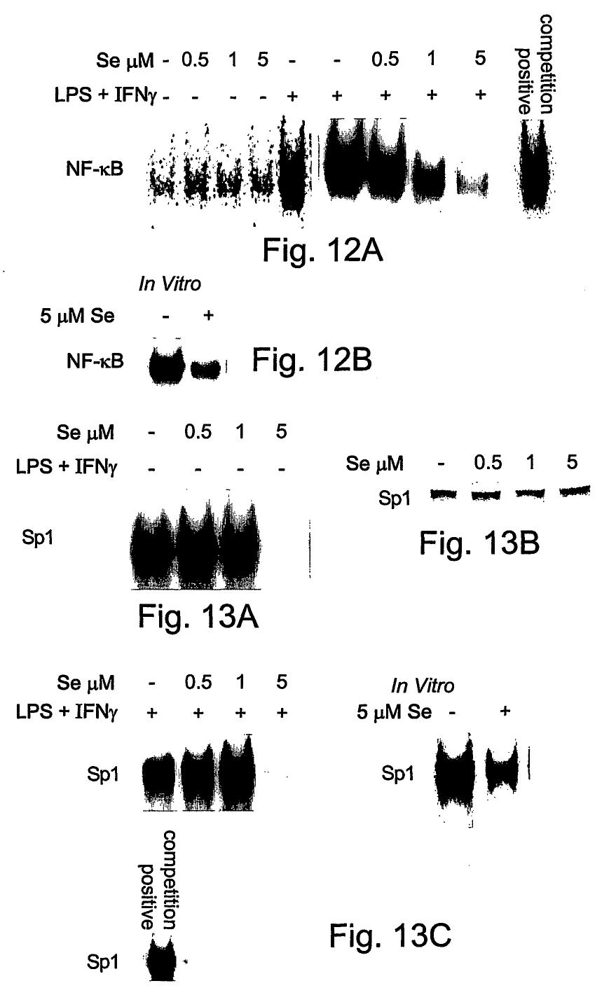

Treatment of macrophages with LPS and INFγ in control macrophages caused an increase in the nuclear import of NF-κB (FIG. 12A, Se+, LPS+INFγ +), and Sp-1 binding (FIG. 13A, middle panel, Se+, LPS+INFγ+), as measured in nuclear extracts by EMSA using specific consensus oligonucleotides selenium treatment dramatically reduced the binding of Sp-1 and nuclear import of NF-κB (FIGS. 12A and 13A, Se+, LPS+INFγ+) in a concentration-dependent manner. Western blot analysis of Sp-1 protein revealed no change in Sp-1 synthesis, indicating a direct effect of selenium on Sp-1 binding of DNA. Further, when the effect of selenium was assessed in a cell-free nuclear extract binding system, it was observed that addition of selenium directly inhibited both NF-κB (FIG. 12B) and Sp-1 (FIG. 13C) binding in vitro. Thus, selenium reduces the activity of two factors central to the regulation of the macrophage radical oxygen species cascade and cytoprotection in inflammatory response.

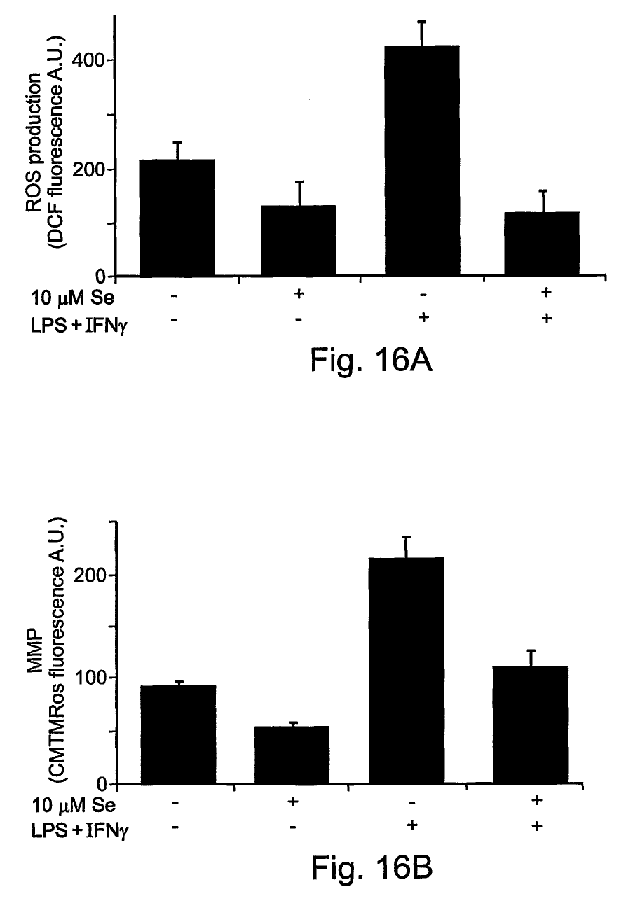

Selenium downregulates Mitochondrial Membrane Potendal and Radical Oxygen Species production in activated macrophages: Activated macrophages, having increased levels of MnSOD, also have higher levels of peroxide production (FIG. 16A, column a), as measured by flow cytometry using intracellular DCF fluorescence. Exposure of the activated macrophages to selenium reduces the levels of oxidation of H₂DCF to fluorescent DCF, indicating inhibition of the MnSOD dismutase activity (FIG. 16A, column c).

The reaction by which MnSOD dismutates superoxide radicals to H₂O₂ requires hydrogen ions. Thus, this reaction helps keep the mitochondrial inner membrane polarized through the removal of protons. To assess the effect that selenium-mediated reduction in MnSOD activity, and the resultant reduced ROS production has on mitochondrial integrity, the mitochondrial inner membrane potential (MMP) was measured in control, activated and selenium-treated, activated J774.2 macrophages by flow cytometry, using the membrane potential-sensitive fluorescent probe CMTMRos.

FIG. 16B shows that, in correlation to the increase in MnSOD, activation of macrophages increases the MMP (column a). Exposure to selenium (10 μM) (column c) downregulates MMP as well as MnSOD in activated macrophages. Thus, in addition to reduction of MnSOD activity and peroxide production, mitochondrial integrity of activated macrophages can be compromised by exposure to selenium.

Selenium-induced cell death in activated macrophages: Exposure to selenium caused the downregulation of MnSOD, peroxide production and reduction of MMP in activated macrophages. In order to determine whether such attenuation of the inflammatory response in macrophages affects the survival of activated immune cells, the effect of selenium on cell viability was assessed by flow cytometry using the nonpermeant DNA-intercalating dye proprium iodide (PI).

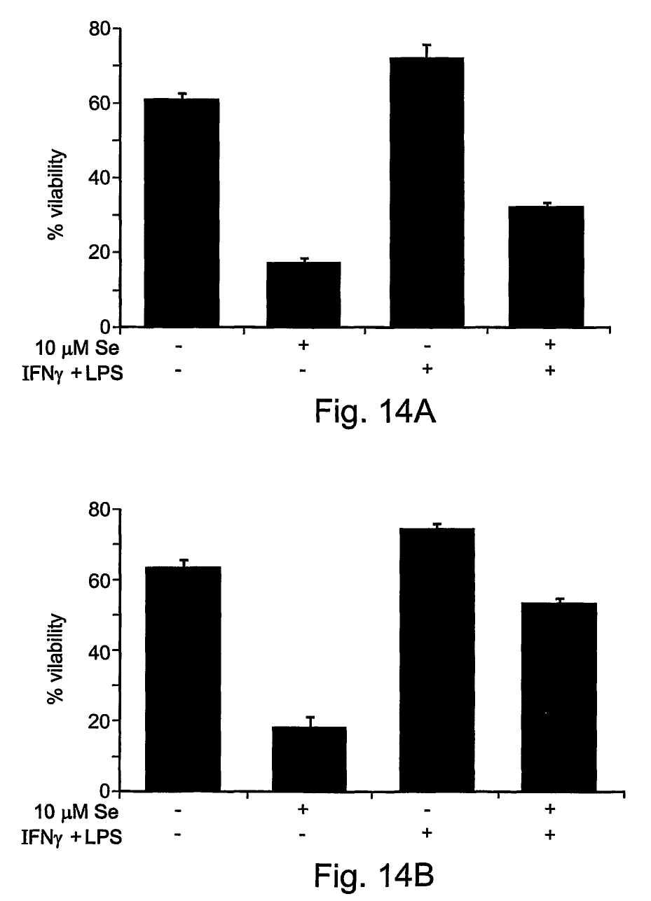

As shown in FIGS. 14A and 14B, activation of J774.2 macrophages for 24 hours with LPS and INFγ significantly increased macrophage viability (column a), whereas 24 hour exposure to selenium (10 μM) decreased viability by 60% in the control cells (column d) and up to 50% in the activated cells (FIG. 14A, column c). It was further observed that the temporal association of the selenium treatment and activation was crucial: when exposure to selenium followed activation by 24 hours, cell survival decreased from 75% to 55% in the treated cells (FIG. 14B, column a compared to column c), whereas simultaneous exposure to selenium and activation of the macrophages resulted in a much greater effect on macrophage survival, from 75% to 35% in the treated cells (FIG. 14A, column a compared to column c).

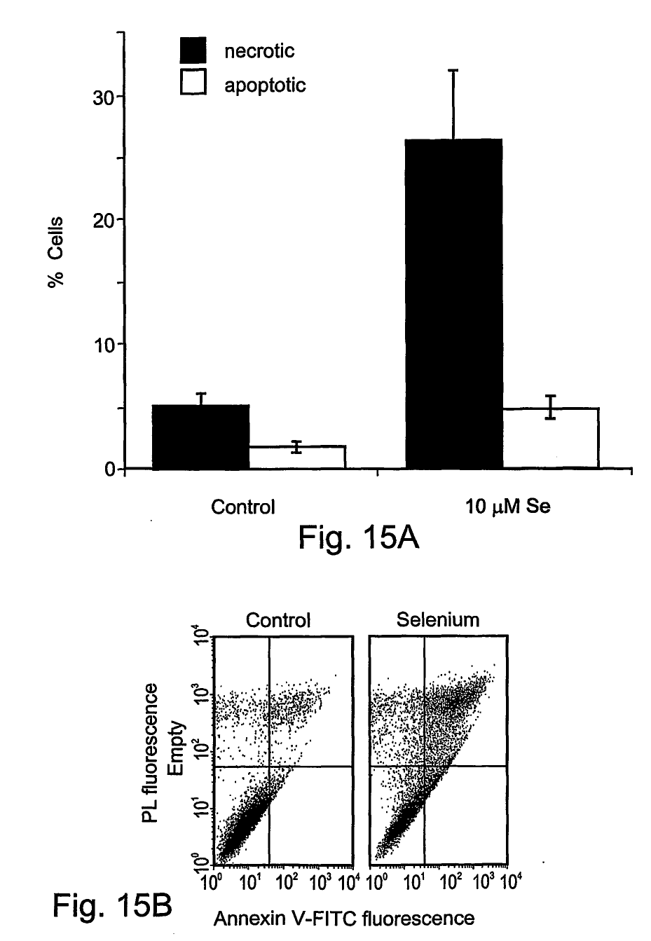

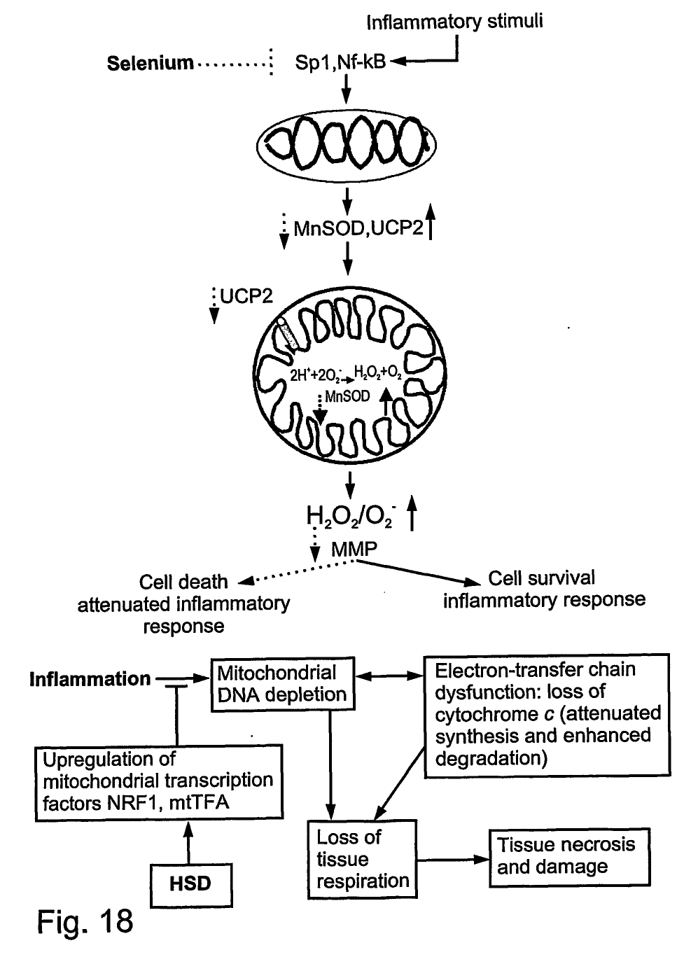

In order to distinguish between necrotic cell death and apoptotic processes resulting from exposure to selenium, the type of cell death was analyzed by flow cytometry using dual staining with annexin V-FITC and PI. Most of the dead macrophages incorporated both PI and annexin V-FITC, indicating cell death with necrotic characteristics (FIG. 14B, quadrant b). No right angle effect of increased incorporation of annexin V-FITC with low PI staining, which would indicate apoptosis, was observed (FIG. 15A, filled column). Without being limited to a single hypothesis, a scheme of the events involved in selenium-induced attenuation of the immune response can be proposed: Selenium sensitizes macrophages, and especially activated macrophages, by affecting transcription factors Sp-1 and nuclear factor NF-κB, resulting in down-regulation of the mitochondrial proteins MnSOD and UCP2, reducing peroxide production and mitochondrial membrane potential, leading to increased inflammatory cell death (FIG. 18).

Taken together, these results show that exposure of activated macrophages to selenium alone can affect the cascade of radical oxygen species production at the level of MnSOD, nuclear factors, compromising mitochondrial inner membrane potential and, ultimately, significantly sensitize the activated macrophages to cell death stimuli. Thus, the protective effects of high dose, dietary selenium supplementation in colitis, and in inflammatory conditions in general, are related to actual reduction in the number, activity and viability of participating inflammatory cells, and attenuation of the inflammatory response, beyond the contribution of selenium to antioxidant selenoprotein complexes.

### Example IV

### Selenium Enhances Mitochondrial Biogenesis in Inflamed and Healthy Tissue

Mitochondrial integrity and function is, as detailed in the Background section hereinabove, crucial to normal cellular metabolism and growth: disruption of mitochondrial biogenesis, or mitochondrial dysfunction has been identified in numerous serious pathologies. Since in-vivo selenium supplementation trials have shown selenium to be actively concentrated in the mitochondria and nuclei, a direct effect of selenium supplementation on mitochondrial factors was investigated.

Selenium prevents inflammation-induced loss of mitochondrial transcription factors by direct action on the affected tissue: FIG. 6 shows that high dose dietary selenium supplementation protects against the loss of colon tissue mitochondrial D-loop DNA in hapten-induced colitis. The D-loop region contains many regulatory sequences for transcription and initiation of replication. FIG. 17 shows the effect of high dose dietary selenium supplementation on mitochondrial transcription factors NRF-1 and mtTFA in colon tissue. Quantitation of the levels of NRF-1 and mtTFA in healthy (FIG. 17, lane 1) and colitis-induced (FIG. 17, lane 3) rats shows the s significant reduction (nearly 50%) in NRF-1 levels, and the nearly absolute loss of mtTFA levels (see graph) in inflamed colon tissue. In tissue from rats receiving high dose dietary selenium supplementation (FIG. 17, lane 4) not only is the loss of NRF-1 and mtTFA prevented, but mitochondrial transcription factor levels even surpass that of healthy control rats (see graph, FIG. 17).

Selenium enhances mitochondrial transcription factors in healthy colon tissue. Surprisingly, measurement of NRF-1 and mtTFA in colon tissue samples from-healthy, selenium treated rats revealed that high dose dietary selenium supplementation significantly enhances tissue levels of NRF-1 and mtTFA beyond those normally found in healthy rats (see graph, FIG. 17), indicating a direct effect of selenium on mitochondrial biogenesis and function, unrelated to effects of TNBS induction of inflammatory colitis.

Taken together, these results show, for the first time that, in addition to protection from macrophage-associated damage in inflamed tissue, high dose dietary selenium supplementation has a direct protective effect on the fully assembled mitochondrial organelle in healthy tissue. As was shown hereinabove, selenium supplementation prevents the loss of mitochondrial respiration and mitochondrial DNA in inflamed tissue (FIGS. 5 and 6, respectively). Without wishing to be limited to a single hypothesis, it is proposed that selenium thus preserves colonic tissue oxygen utilization for energy requirements, attenuating necrotic damage in the inflamed tissue (see FIG. 18). Indeed, high dose dietary selenium supplementation did not enhance mitochondrial activity, i.e. respiration rate, beyond that found in the control animals (FIG. 5), similar to the normal mitochondrial activity found in NRF1 transgenic animals. Thus, whereas increased expression of this transcription factor may not necessarily result in increased mitochondrial activity beyond that needed for the energy requirements of the tissue, depletion of NRF-1, as in IBD and other inflammatory conditions, can lead to the loss of tissue mitochondrial function and oxygen utilization and, ultimately, necrosis of the effected tissue. Further, in view of the above, high dose selenium supplementation can be used as a novel treatment in the prevention of diseases and conditions associated with alteration of mitochondrial function and biogenesis.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by, for example, gene bank accession numbers, mentioned in this specification are herein incorporated in their entirety by reference into the specification, to the same extent as if each individual publication, patent, patent application or sequence was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

Amer, E. S., and Holmgren, A. (2000). Physiological functions of thioredoxin and thioredoxinreductase, Eur J Biochem 267, 6102-9.

Beattie, R. M., Schiffrin, E. J., Donnet-Hughes, A., Huggett, A. C., Domizio, P., MacDonald, T. T., and Walker-Smith, J. A. (1994). Polymeric nutrition as the primary therapy in children with small bowel Crohn's disease, Aliment Pharmacol Ther 8, 609-15.

Briet, F., Twomey, C., and Jeejeebhoy, K. N. (2003). Effect of malnutrition and short-term refeeding on peripheral blood mononuclear cell mitochondrial complex I activity in humans, Am J Clin Nutr 77, 1304-11.

Brigelius-Flohe, R., Maurer, S., Lotzer, K., Bol, G., Kallionpaa, H., Lehtolainen, P., Viita, H., and Yla-Herttuala, S. (2000). Overexpression of PHGPx inhibits hydroperoxide-induced oxidation, NFkappaB activation and apoptosis and affects oxLDL-mediated proliferation of rabbit aortic smooth muscle cells, Atherosclerosis 152, 307-16.

Davis, C. D., Brooks, L., Calisi, C., Bennett, B. J., and McElroy, D. M. (1998). Beneficial effect of selenium supplementation during murine infection with Trypanosoma cruzi, J Parasitol 84, 1274-7.

Delpre, G., Avidor, I., Steinherz, R., Kadish, U., and Ben-Bassat, M. (1989). Ultrastructural abnormalities in endoscopically and histologically normal and involved colon in ulcerative colitis, Am J Gastroenterol 84, 1038-46.

Geerling, B. J., Badart-Smook, A., Stockbrugger, R. W., and Brummer, R. J. (2000). Comprehensive nutritional status in recently diagnosed patients with inflammatory bowel disease compared with population controls, Eur J ClinNutr 54, 514-21.

Green, D. R., and Reed, J. C. (1998). Mitochondria and apoptosis, Science 281, 1309-12.

Hirsch, T., Susin, S. A., Marzo, I., Marchetti, P., Zamzami, N., and Kroemer, G. (1998). Mitochondrial permeability transition in apoptosis and necrosis, Cell Biol Toxicol 14, 141-5.

Korsmeyer, S. J., Wei, M. C., Saito, M., Weiler, S., Oh, K. J., and Schlesinger, P. H. (2000). Pro-apoptotic cascade activates BID, which oligomerizes BAK or BAX into pores that result in the release of cytochrome C, Cell Death Differ 7, 1166-73.

Krawisz, J. E., Sharon, P., and Stenson, W. F. (1984). Quantitative assay for acute intestinal inflammation based on myeloperoxidase activity. Assessment of inflammation in rat and hamster models, Gastroenterology 87, 1344-50.

Kroemer, G., Dallaporta, B., and Resche-Rigon, M. (1998). The mitochondrial death/life regulator in apoptosis and necrosis, Annu Rev Physiol 60, 619-42.

Kroemer, G., Zanzami, N., and Susin, S. A. (1997). Mitochondrial control of apoptosis, Immunology Today 18, 44-51.

Lossos, A., River, Y., Eliakim, A., and Steiner, I. (1995). Neurologic aspects of inflammatory bowel disease, Neurology 45, 416-21.

Moum, B. (2000). Medical treatment: does it influence the natural course of inflammatory bowel disease?, Eur J Intern Med 11, 197-203.

Nicholls, R. J. (2002). Review article: ulcerative colitis—surgical indications and treatment, Aliment Pharmacol Ther 16Suppl 4, 25-8.

Podolsky, D. (2000a). Pride and prejudice: inflammatory bowel disease models and drug development, current opinions in gastroenterology 16,295-6.

Podolsky, D. K. (2000b). Going the Distance—The Case for True Colorectal-Cancer Screening, N Engl J Med 343, 207-208.

Reifen, R, Matas, Z., Zeidel, L., Berkovitch, Z., and Bujanover, Y. (2000). Iron supplementation may aggravate inflammatory status of colitis in a rat model, Dig Dis Sci 45, 394-7.

Ringstad, J., Kildebo, S., and Thomassen, Y. (1993). Serum selenium, copper, and zinc concentrations in Crohn's disease and ulcerative colitis, Scand J Gastroenterol 28, 605-8.

Shilo, S., Aronis, A., Komarnitsky, R., and Tirosh, O. (2003). Selenite sensitizes mitochondrial permeability transition pore opening in vitro and in vivo: a possible mechanism for chemo-protection, Biochem J 370, 283-90.

Shilo, S., and Tirosh, O. (2003). Selenite activates caspase-independent necrotic cell death in Jurkat T Cells and J774.2 macrophages by affecting mitochondrial oxidant generation., Antioxidants & Redox Signaling in press.

Thirunavukkarasu, C., and Sakthisekaran, D. (2003). Influence of sodium selenite on glycoprotein contents in normal and N-nitrosodiethylamine initiated and phenobarbital promoted rat liver tumors, Pharmacol Res 48, 167-73.

Valacchi, G., Weber, S. U., Luu, C., Cross, C. E., and Packer, L. (2000). Ozone potentiates vitamin E depletion by ultraviolet radiation in the murine stratum corneum, FEBS Lett 466, 165-8.

Wewer, V., Gluud, C., Schlichting, P., Burcharth, F., and Binder, V. (1991). Prevalence of hepatobiliary dysfunction in a regional group of patients with chronic inflammatory bowel disease, Scand J Gastroenterol 26, 97-102.

1-61. (canceled)

62. A method for treating a subject having an inflammatory disease or condition comprising administering to the subject selenium in a daily amount of about 0.1 μmol to about 100 μmol per kg body weight, for at least one day, thereby treating the subject for the inflammatory disease or condition.

63. The method of claim 62, wherein said administering is for not more than 21 days.

64. The method of claim 62, wherein said administering is for not more than 12 months.

65. The method of claim 62, wherein said daily amount is about 2 μmol to about 50 μmol per kg body weight.

66. The method of claim 62, wherein said daily amount is about 10 μmol to about 20 μmol per kg body weight.

67. The method of claim 62, wherein said selenium is administered as a constituent of a selenium source.

68. The method of claim 67, wherein said selenium source is selected from the group consisting of an inorganic selenium compound and an organoselenium compound.

69. The method of claim 62, wherein said inflammatory disease is selected from the group consisting of hypersensitivity, an autoimmune disease, an infectious disease, graft rejection, an allergic disease, an inflammatory musculo-skeletal disease, a gut-related inflammatory disease, a neurological inflammatory disease, an inflammatory cardiovascular disease, an injury, an idiopathic inflammatory disease and an inflammation of unknown etiology.

70. The method of claim 62, wherein said administering is carried out twice daily.

71. A method for treating a subject having a macrophage-mediated inflammatory disease or condition comprising administering to the subject selenium in a daily amount of about 0.1 μmol to about 100 μmol per kg body weight, for at least one day, thereby treating the subject for the macrophage-mediated inflammatory disease or condition.

72. The method of claim 71, wherein said macrophage-mediated inflammatory disease is selected from the group consisting of atherosclerosis, glomerulonephritis, histiocytosis and Guillain-Barre syndrome.

73. A method for downregulating an activity of an inflammatory immune cell, the method comprising exposing the cell to a concentration of selenium sufficient to downregulate an inflammatory process in the cell, thereby downregulating an activity of the inflammatory immune cell.

74. The method of claim 73, wherein said concentration of selenium is about 0.1 μM to about 1000 μM selenium.

75. The method of claim 74 wherein said concentration of selenium is about 0.5 μM to about 50 μM.

76. The method of claim 73, wherein said exposing the cell is performed in vivo or in vitro.

77. The method of claim 73, wherein said inflammatory process comprises production of reactive oxygen species and whereby said downregulation is via reduced expression of mitochondrial proteins.

78. The method of claim 73, wherein said inflammatory immune cell is selected from the group consisting of T-lymphocytes, dendritic cells, eosinophils, macrophages, granulocytes, monocytes and macrophages.

79. A method for treating a subject having a disease or condition associated with altered mitochondrial function comprising administering to the subject selenium in a daily amount of about 0.1 μmol to about 100 μmol per kg body weight, for at least one day, thereby treating the subject for the disease or condition.

80. The method of claim 79, wherein said administering is for not more than 21 days.

81. The method of claim 79, wherein said administering is for not more than 12 months.

82. The method of claim 79, wherein said daily amount is about 2 μmol to about 50 μmol per kg body weight.

83. The method of claim 79, wherein said daily amount is about 10 μmol to about 20 μmol per kg body weight.

84. The method of claim 79, wherein said selenium is administered as a constituent of a selenium source.

85. The method of claim 84, wherein said selenium source is selected from the group consisting of an inorganic selenium compound and an organoselenium compound.

86. The method of claim 79, wherein said disease or condition associated with altered mitochondrial function is selected from the group consisting of Alzheimer's Disease, Parkinson's Disease, Huntington's disease, progressive supranuclear palsy, diabetes mellitus, hyperproliferative disorders such as cancer, tumors and psoriasis, amyotrophic lateral sclerosis (ALS), Friedreich's ataxia, colon cancer, stroke, exercise intolerance and cardiac myopathy.

87. A selenium solid oral dosage form unit comprising as an active ingredient from about 0.005 to about 10 mmol of selenium in a volume of less than 1 cm³ and a pharmaceutically acceptable carrier.

88. The solid oral unit dosage of claim 87, wherein said selenium dosage form unit comprises about 0.05 mmol to about 5 mmol selenium.

89. The solid oral unit dosage of claim 87, wherein said selenium dosage form unit comprises about 0.5 mmol to about 2.5 mmol selenium.

90. The solid oral unit dosage of claim 87, wherein said pharmaceutical composition comprises selenium as a constituent of a selenium source.

91. The solid oral unit dosage of claim 90, wherein said selenium source is selected from the group consisting of an inorganic selenium compound and an organoselenium compound.

92. An article of manufacture comprising a packaging material and at least one selenium dosage form unit, said selenium dosage form unit comprising a pharmaceutical composition comprising from about 0.005 to about 10 mmol selenium and a pharmaceutically acceptable carrier in each single unit dosage, and wherein said packaging material comprises a label or package insert indicating that said pharmaceutical composition is for treating and/or preventing an inflammatory disease or condition.

93. The article of manufacture of claim 92, wherein said selenium dosage form unit comprises about 0.05 mmol to about 5 mmol selenium.

94. The article of manufacture of claim 92, wherein said selenium dosage form unit comprises about 0.5 mmol to about 2.5 mmol selenium.

95. The article of manufacture of claim 92, wherein said pharmaceutical composition comprises selenium as a constituent of a selenium source.

96. The article of manufacture of claim 95, wherein said selenium source is selected from the group consisting of an inorganic selenium compound and an organoselenium compound.

97. The article of manufacture of claim 92, wherein said composition is in the form of a liquid dosage form.
